(19) United States
(12) Patent Application Publication
(10) Pub. No.: US 2025/0221954 A1
(43) Pub. Date: Jul. 10, 2025

(54) METHODS TO TREAT, PREVENT, OR REDUCE HYPERAMMONEMIA

(71) Applicant: Darren Alexander Rubin, Largo, FL (US)

(72) Inventor: Darren Alexander Rubin, Largo, FL (US)

(21) Appl. No.: 18/978,528

(22) Filed: Dec. 12, 2024

Related U.S. Application Data
(60) Provisional application No. 63/651,538, filed on May 24, 2024.
(60) Provisional application No. 63/618,443, filed on Jan. 8, 2024.

(51) Int. Cl. A61K 31/222; A61K 31/165; A61K 31/192; A61K 31/197; A61K 31/662; A61K 38/45; A61K 38/53

(52) U.S. Cl. CPC A61K 31/222; A61K 31/165; A61K 31/192; A61K 31/197; A61K 31/662; A61K 38/45; A61K 38/53; C12Y 203/01014; C12Y 203/01071; C12Y 603/01002

(57) ABSTRACT

Methods, formulations, and applications of treating and preventing hyperammonemia by reducing ammonia levels are provided, which also have implications for reducing hemodialysis.

### CROSS-REFERENCE TO RELATED APPLICATIONS

This patent application claims priority to U.S. Provisional Application Ser. No. 63/651,538 filed May 24, 2024, entitled “Methods to Treat, Prevent, or Reduce Hyperammonemia,” and U.S. Provisional Application Ser. No. 63/618,443 filed Jan. 8, 2024, entitled “Methods to Treat, Prevent, or Reduce Hyperammonemia,” which is hereby incorporated by reference in their entirety into the present disclosure.

### BACKGROUND

Hyperammonemia is a life-threatening, metabolic condition of very elevated ammonia levels that can cause brain damage, ataxia, seizures, coma, and even death. Ammonia, or its ionized form ammonium, crosses cellular membranes, as well as, the blood brain barrier. High levels of ammonia in the brain are neurotoxic; ammonia swells astrocytes, causes neuronal cell death, compromises blood-brain barrier permeability, and causes cerebral edema. Seizures often result from ammonia's interference with inhibitory neurotransmission.

While there are various causes of hyperammonemia, there are two main groups of causes, urea cycle disorders and liver disease, although these conditions can be interlinked. Liver disease may be acute or chronic. When liver disease is associated with elevated ammonia levels and neurological dysfunction, the condition is called hepatic encephalopathy and can lead to confusion, disorientation, coma, and even death. Liver disease itself, such as that caused by an infection or an exposure to a toxin, may also impair the urea cycle; while urea cycle disorders may also impair and damage the liver.

The urea cycle is the body's primary mechanism for removal of nitrogenous waste. There exist a series of metabolic reactions that produce the nitrogenous compound urea from ammonia. Urea is an amide with two amino groups which is excreted in the urine. Each mol of urea excreted is the equivalent of the nitrogenous waste removal of two mols of ammonia. Urea cycle disorders are rare diseases involving genetic defects in any of the six enzymes, or any of the two transporters, involved in the urea cycle, and may manifest shortly after birth so that newborns are especially vulnerable. Ornithine transcarbamylase (OTC) deficiency is the most common urea cycle disorder, while others include carbamoyl phosphate synthetase 1 (CPS1) deficiency, N-acetylglutamate synthase (NAGS) deficiency, arginase deficiency (ARG deficiency), argininosuccinate synthetase 1 (ASS1) deficiency (citrullinemia), and argininosuccinate lyase (ASL) deficiency (arginosuccinic acidemia). Cirrhosis may develop when the urea cycle disorder affects the liver. Nearly all of the seven or so individual disorders comprising urea cycle disorder are associated with hyperammonemia, although a couple disorders may instead present with argininemia. Mild forms of urea cycle disorder, such as partial enzyme deficiency, may manifest later in life and require strict dietary management of protein and amino acid intake.

The management of urea cycle disorders include restricting dietary protein, as well as taking oral medications of ammonia scavengers, such as phenylbutyrate, to lower ammonia. For certain urea cycle disorders, supplementation with urea cycle intermediates, such as arginine or citrulline amino acids are helpful. If severe, hemodialysis may be needed. Metabolic decompensation may occur if medication is not taken or there is too much protein intake, as well as, if infection or liver dysfunction occurs. Although not very common, gastrointestinal bleeding or menstruation may cause a relapse.

Non-genetic causes of urea cycle disorder also result in hyperammonemia and include liver damage, exposure to chemical toxins and alcohol, infections, and other metabolic diseases. Certain drugs can have a direct interference with these urea cycle enzymes, including valproic acid and some chemotherapies; while other drugs can damage liver function, including acetaminophen overdose, other chemotherapies, as well as, systemic antifungal medications.

Hyperammonemia can also be caused by other conditions, including a portosystemic shunt, surgical procedures such as heart or lung transplant, kidney disease, and traumatic bleeding. Even too much parenteral nutrition may lead to nitrogen accumulation.

Normal blood ammonia levels may vary with age. Typically, ammonia levels are below 50 micromol per liter, but the upper limit of normal for children and adults can reach as high as 80 micromol per liter. For neonates, the upper limit of normal can reach as high as 110 micromol per liter. Ammonia is considered elevated above these values, and dangerously high if above 200 micromol per liter. It is essential to bring blood levels of ammonia down during a hyperammonemia crisis to avoid permanent neurological damage or death.

Hemodialysis, a form of renal replacement therapy, is a rapid method to remove elevated ammonia from the blood, however, hemodialysis is not without its own risks and complications; especially to newborns and young children having small blood flow volumes. Hemodialysis may result in sepsis, aneurysm, hemorrhage, embolism, cardiac arrhythmia, or even aortic dissection and myocardial infarction. Hemodialysis may need to be repeated until stabilization of ammonia levels. Clearance of ammonia by hemodialysis is approximately ten times greater than by peritoneal dialysis or hemofiltration.

There exist pharmacologic treatments to reduce blood ammonia levels, including intravenous drug infusions, as well as, oral medications. However, these pharmacologic treatments only postpone or avoid hemodialysis some of the time. For patients who are less responsive to these pharmacologic treatments, such infusions may be maintained for multiple days as this treatment may be slow and inefficient.

### BRIEF SUMMARY

Various embodiments are directed to a pharmaceutical preparation comprising at least one of: a phenylbutyrate ester or a phenylbutyrate amide, wherein the phenylbutyrate ester or the phenylbutyrate amide does not hydrolyze into glycerol during hydrolyzation to release phenylbutyrate molecules as a prodrug nitrogen-binding agent for managing nitrogen levels, treating hyperammonemia, preventing hyperammonemia, treating hepatic encephalopathy, preventing hepatic encephalopathy, or a combination thereof.

In certain embodiments, said pharmaceutical preparation increases a receiving body's levels of at least one of triglyceride, lipoprotein particle, total cholesterol, body fat, or a combination thereof after hydrolysis by an amount less than an increase caused by an application of glycerol to the body. In various embodiments, the pharmaceutical preparation further comprises at least one of ornithine, glutamate, glutamyl-phosphate, benzoate, ornithine benzoate ester, glutamate benzoate ester, phenylbutyrate benzoate ester, ornithine phenylbutyrate ester, glutamate phenylbutyrate ester, ornithine benzoate amide, glutamate benzoate amide, ornithine phenylbutyrate amide, glutamate phenylbutyrate amide, diphenylbutyrate ester, or dibenzoate ester. In certain embodiments, the phenylbutyrate ester and or phenylbutyrate amide is a molecule covalently bonded to at least one of benzoate, ornithine, glutamate, or a derivative, or combination thereof.

Certain embodiments are directed to a pharmaceutical preparation comprising: at least one molecule that when hydrolyzed releases both: at least one phenylbutyrate molecule and at least one molecule, wherein the at least one molecule is at least one of benzoate, ornithine, glutamate, or a derivative, or combination thereof; wherein the at least one phenylbutyrate molecule is released as a prodrug nitrogen-binding agent for managing nitrogen levels, treating hyperammonemia, preventing hyperammonemia, treating hepatic encephalopathy, preventing hepatic encephalopathy, or a combination thereof.

Certain embodiments are directed to a pharmaceutical preparation comprising at least one phenylbutyrate amide that hydrolyzes to release phenylbutyrate molecules as a prodrug nitrogen-binding agent for managing nitrogen levels, treating hyperammonemia, preventing hyperammonemia, treating hepatic encephalopathy, preventing hepatic encephalopathy, or a combination thereof.

In certain embodiments, the at least one of: a phenylbutyrate ester or a phenylbutyrate amide is provided in an amount of at least one milligram, a volume of at least one milliliter, a concentration of at least one milligram per milliliter, or a combination thereof, of said at least one phenylbutyrate ester and or phenylbutyrate amide. In various embodiments, said at least one phenylbutyrate ester comprises a glycol (or diol) esterified to two 4-phenylbutanoate groups. In certain embodiments, said at least one phenylbutyrate ester comprises propane-1,2-diyl bis(4-phenylbutanoate); propane-1,3-diyl bis(4-phenylbutanoate); butane-1,3-diyl bis(4-phenylbutanoate); or a combination thereof. In various embodiments, said at least one phenylbutyrate ester comprises PEGylated phenylbutyrate and or PEGylated phenylbutyrate esters. In certain embodiments, the pharmaceutical preparation is provided as a composition for one of: oral administration, administration via a feeding tubing, administration via a gastrostomy tube, administration via a nasogastric tube, or rectal administration. In certain embodiments, the pharmaceutical composition further comprises at least one excipient ingredient selected from: flavoring agents, flavorants, sweeteners, taste-masking agents, solvents, tonicity agents, chelators, buffers, pH adjusting agents, solubility enhancing agents, absorption enhancing agents, cell membrane permeability enhancing agents, excipients for delaying release or delaying absorption, preservatives, or a combination thereof. In various embodiments, the pharmaceutical preparation further comprises at least one recombinant enzyme or enzyme expression system thereof, including at least one of glutamine synthetase, glutamine phenylacetyltransferase, glycine benzoyltransferase, or a combination thereof, to increase at least one enzymatic activity in a patient's body. In certain embodiments, the pharmaceutical preparation is provided as a composition for injection, and wherein the pharmaceutical preparation comprises at least one sterile container selected from prefilled syringes, cartridges, autoinjectors, pen injectors, wearable injectors, portable infusion pump devices, vials, ampoules, intravenous fluid bags, and bottles. In some embodiments, the pharmaceutical preparation further comprises at least one additional active ingredient used to treat a urea cycle disorder, liver disease, hyperammonemia, hepatic encephalopathy, or a combination thereof.

Certain embodiments are directed to a pharmaceutical preparation comprising at least one of ornithine benzoate ester, glutamate benzoate ester, phenylbutyrate benzoate ester, ornithine phenylbutyrate ester, glutamate phenylbutyrate ester, ornithine benzoate amide, glutamate benzoate amide, ornithine phenylbutyrate amide, glutamate phenylbutyrate amide, or a combination thereof that hydrolyzes to release one or more of: (a) phenylbutyrate molecules or (b) benzoate molecules as a nitrogen-binding agent for managing nitrogen levels, treating hyperammonemia, preventing hyperammonemia, treating hepatic encephalopathy, preventing hepatic encephalopathy, or a combination thereof.

Certain embodiments are directed to a method comprising: administering at least one dosage of at least one pharmaceutical preparation comprising at least one of: a phenylbutyrate amide or a phenylbutyrate ester other than 2,3-bis(4-phenylbutanoyloxy)propyl 4-phenylbutanoate that hydrolyzes to release phenylbutyrate molecules as a prodrug nitrogen-binding agent for managing nitrogen levels, treating hyperammonemia, preventing hyperammonemia, treating hepatic encephalopathy, preventing hepatic encephalopathy, or a combination thereof; wherein said method causes at least one of: levels of circulating phenylacetate or phenylacetylglutamine to increase by more than 1 percent in a body of a patient following administration, levels of phenylacetylglutamine excreted by kidneys in the body of the patient to increase by more than 1 percent in the body of the patient following administration, or levels of circulating ammonia or ammonium ion to decrease by more than 1 percent in the body of the patient following administration.

In certain embodiments, the method further comprises administering at least one of ornithine, benzoate, ornithine benzoate, sodium benzoate, glutamate, glutamate benzoate, glutamyl-phosphate, or a salt, prodrug, derivative, or combination thereof in said at least one pharmaceutical preparation or separately. In certain embodiments, the method further comprises administering said at least one dosage to a patient with a urea cycle disorder; to a patient with an inborn error of metabolism, enzyme deficiency, and or enzyme inefficiency; to a patient with an acute liver disease or an acute liver failure; to a patient with a chronic liver disease or a chronic liver failure or liver cirrhosis or liver transplant; or a combination thereof. In various embodiments, the method further comprises administering said at least one dosage to a patient, wherein the patient satisfies one or more of: (a) the patient has high levels of at least one of triglycerides, lipoprotein particles, cholesterol, body fat, or a combination thereof; or (b) the patient has or is close to having at least one of heart disease, cardiovascular disease, atherosclerosis, lipid storage disease, or a combination thereof; for which glycerol intake should be reduced, restricted, or contraindicated. In certain embodiments, the method further comprises steps of measuring blood or plasma ammonia or ammonium levels at least once before or at least once after administering said an at least one dosage to said patient at least once. In various embodiments, the method further comprises one or more of: sequencing a patient's genes; detecting genetic mutations in the patient; or measuring levels of activity of one or more enzymes involved in and or related to metabolism of glutamate, glutamine, ornithine, alpha-ketoglutarate, ammonia, urea, benzoate, phenylacetate, or a combination thereof.

In various embodiments, the method further comprises administering at least one biotechnology formulation that includes one or more recombinant enzymes, including at least one of glutamine synthetase, glutamine phenylacetyltransferase, glycine benzoyltransferase, or a combination or expression system thereof. In certain embodiments, said an at least one pharmaceutical preparation further comprises at least one biotechnology formulation that includes one or more recombinant enzymes, including at least one of glutamine synthetase, glutamine phenylacetyltransferase, glycine benzoyltransferase, or a combination or expression system thereof.

Various embodiments are directed to a biotechnology formulation for injection or infusion comprising one or more recombinant enzymes for injection, including at least one of glutamine synthetase for injection, glutamine phenylacetyltransferase for injection, glycine benzoyltransferase for injection, or a combination thereof, and at least one excipient ingredient.

Certain embodiments are directed to a method, comprising: administering at least one biotechnology formulation for injection or infusion that comprises at least one recombinant enzyme for injection, including at least one of glutamine synthetase for injection, glutamine phenylacetyltransferase for injection, glycine benzoyltransferase for injection, or a combination thereof, for treating hyperammonemia, preventing hyperammonemia, treating hepatic encephalopathy, preventing hepatic encephalopathy, or a combination thereof.

In certain embodiments, the method further comprises a step of administering at least one substrate or active ingredient including at least one of glutamate, glutamyl-phosphate, phenylacetate, phenylbutyrate, benzoate, ornithine, ornithine benzoate ester or amide, glutamate benzoate ester or amide, phenylbutyrate ester, phenylbutyrate amide, or a salt, prodrug, derivative, or combination thereof.

### DETAILED DESCRIPTION

The following description details methods of treating, preventing, or reducing hyperammonemia, as well as, its symptoms and reoccurrence. These methods include new and improved pharmacologic treatments and or biotechnology treatments. There exists a great need for safely and rapidly decreasing elevated ammonia levels in patients, including patients having urea cycle disorders, liver disease, infection, exposure to certain drug(s) and or toxin(s), or a combination thereof. This includes patients having hyperammonemia, to quickly reduce further damage caused by elevated ammonia levels and to avoid hemodialysis. This also includes patients having difficulty managing elevated ammonia levels so as to prevent a hyperammonemia crisis.

A standard of care treatment for hyperammonemia is the drug product brand Ammonul®, and its generics, consisting of sodium phenylacetate 10% and sodium benzoate 10% solution for intravenous infusion. Ammonul is supplied in a 50 mL vial containing 5 grams each of sodium phenylacetate and sodium benzoate. The labeling for Ammonul states that it is indicated as adjunctive therapy in pediatric and adult patients for the treatment of acute hyperammonemia and associated encephalopathy in patients with deficiencies in enzymes of the urea cycle. Ammonul must be diluted in sterile 10% dextrose injection before administration, which also provides calories in the form of glucose. While Ammonul received FDA approval in 2005, clinical studies began decades prior and the concept of Ammonul was developed before 1920. Ammonul is administered as a loading (bolus) dose over 1.5 hours to 6 hours, but usually for up to two hours, followed by maintenance dosing continued for 24-hour periods until the patient is no longer hyperammonemic or oral therapy could be tolerated. In worsening conditions, bolus dosing may be used again. Dosing of Ammonul depends on the patient's body weight. Patients weighing up to 20 kilograms generally receive a loading dosage of 2.5 mL per kilogram of body weight, followed by maintenance dosing over 24 hours. Patients over 20 kilograms generally receive a loading dosage of 55 mL per meters squared (which is a measure of body surface area), followed by maintenance dosing over 24 hours.

Intravenous arginine may also be provided in these episodes as it is an essential component of therapy for patients with CPS, OTC, ASS, or ASL deficiency.

One active ingredient in Ammonul is sodium benzoate. A mitochondrial-based, acyl-coenzyme A transferase enzyme, one known as glycine benzoyltransferase, is believed to acylate the benzoate molecule, forming a benzoyl coenzyme A intermediate, which then combines with the amino acid glycine to form hippurate. The coenzyme A (abbreviated as CoA) is recycled and energy in the form of adenosine triphosphate (ATP) is used in this process. Importantly, the hippurate is cleared by the kidneys and excreted in the urine. In effect, glycine is removed from the body; glycine being an amino acid having one amino group is a nitrogenous molecule.

The other active ingredient in Ammonul is sodium phenylacetate. Again, a mitochondrial-based, acyl-CoA transferase enzyme, one known as glutamine phenylacetyltransferase, is believed to acylate the phenylacetate molecule, which then combines with the amino acid glutamine to form phenylacetylglutamine. The CoA is recycled and energy in the form of ATP is used in this process. Importantly, the phenylacetylglutamine is cleared by the kidneys and excreted in the urine. In effect, glutamine is removed from the body; glutamine being an amino acid having two amino groups is a nitrogenous molecule. Glutamine has double the nitrogen compared to glycine. With two nitrogen atoms, glutamine has as much nitrogen as urea has.

Glutamine is much more abundant than glycine; glutamine being the most abundant free amino acid in the blood, constituting 20% to 25% of these free amino acids. Glutamine serves a prominent role in the transport of carbon and nitrogen throughout the body and between organs. Glutamine also provides a route for the transport of glutamate (and nitrogen) out of the brain. Glutamine has many uses in the body, not least as a precursor for purine and pyrimidine synthesis, and as a precursor for the antioxidant glutathione. Glutamine also serves as oxidative fuel for rapidly dividing cells such as immune cells, fibroblasts, and enterocytes. Glutamine also serves as an intermediate for gluconeogenesis as it is catabolized into the Kreb cycle intermediate, alpha-ketoglutarate. Glutamine is a cellular energy source secondly to glucose. Glutamine also donates an amide nitrogen via various amidotransferase reactions needed to make many nitrogen-containing molecules.

The liver is the main site of glutamine catabolism and production of urea for excretion by the kidneys; the urea cycle and urea being the main mechanism of nitrogenous waste removal. Each mol of urea has the equivalent of two mols of nitrogen (two mols of ammonia). Besides the excretion of urea, under normal conditions, the kidneys also play another role in ammonia excretion, although this is only about one-tenth that of urea. This other process is called renal ammoniagenesis whereby the kidneys catabolize glutamine into two mols of ammonia and two mols of bicarbonate. Renal ammoniagenesis, however, becomes much more important during conditions of metabolic acidosis, whereby the ammonium helps in the excretion of acids and the bicarbonate is provided to the bloodstream to help neutralize the acidosis.

Since dietary glutamine (and glutamate) is generally catabolized by intestinal epithelial cells before reaching the blood, and any remaining untouched glutamine is catabolized by the liver, most of the glutamine found in the body is synthesized de novo. This system may be saturable and in some instances for example, multi-gram dose(s) of oral glutamate may achieve high peak plasma levels transiently for one or more hours. Main sites for glutamine synthesis and release is in muscles, as well as, adipose tissue and lungs. The liver has the capacity to both use and make glutamine. The only enzyme responsible for glutamine synthesis is glutamine synthetase, which is an ATP-dependent enzyme that forms glutamine from the amino acid glutamate and ammonia. Glutamine synthetase catalyzes a two-step reaction: firstly using ATP to phosphorylate glutamate into an acyl-phosphate intermediate called glutamyl-phosphate (or gamma-glutamyl phosphate); and secondly whereby free ammonia as a nucleophile then replaces the phosphate group to form the nitrogen of glutamine's amide side chain. The process of glutamine synthesis sequesters one ammonia for each glutamine molecule made. The main function of glutamine synthetase in many tissues is the removal of toxic ammonia. By sequestering ammonia and converting glutamate into glutamine, glutamine synthetase serves as a key regulator of nitrogen metabolism; controlling the uptake of nitrogen where needed and removing nitrogen when elevated to alleviate its toxicity. The glutamine made can be sent off into the blood stream for interorgan transport, including for transport to the liver for urea production and subsequent nitrogen removal by the kidneys. The glutamine pool in the blood plasma turns over rapidly.

Glutamine synthetase is found in many areas of the body including the brain, liver, adipose tissue and lung, but is especially prominent in muscle tissue where nearly three-fourths of endogenous glutamine production occurs. Glutamine synthesis in muscle tissue may become more important when blood levels of glutamine fall, or when blood levels of ammonia rise.

Glutamine synthetase is also expressed in endothelial cells, such as blood vessels, but glutamine production is believed negligible during normal conditions. Glutamine synthetase is a cytosolic enzyme. Yet, this enzyme was found to palmitoylate itself and localize to cell membranes, for signaling purposes for processes such as angiogenesis.

Once glutamine is made, it can be used where it was made or be transported via the vasculature to other areas of the body for other uses and needs, including for protein or nucleotide synthesis, or to the liver or kidney for nitrogen excretion. Just as ammonia was added to make glutamine, the amino group can be transferred in various transaminase reactions, or can come off to form ammonia again. Glutaminase is the amidohydrolase enzyme that creates glutamate from glutamine while releasing ammonia. Glutaminase is a mitochondrial enzyme mainly expressed in the liver to generate ammonia (ammonium) for urea synthesis, and is also expressed in the kidneys to excrete ammonium ions, but more so during acidosis. The glutamate produced may be a feedback inhibitor of the glutaminase reaction.

Glutamate formed by glutaminase can then be used or can form other amino acids such as aspartate or alanine via transaminase enzymes, which include alanine aminotransaminase (abbreviated as ALT) and aspartate aminotransferase (abbreviated as AST). While ALT and AST are found in the liver and some other areas of the body, these enzymes can leak out of damaged cells and are detected in blood tests, which may indicate liver damage when elevated.

Or, the glutamate that glutaminase generates can form ammonia and alpha-ketoglutarate via the mitochondrial enzyme, glutamate dehydrogenase, for which alpha-ketoglutarate can enter the Krebs cycle to produce energy in the form of ATP. In mammals, glutamate dehydrogenase equilibrium favors the production of ammonia and alpha-ketoglutarate, rather than the reverse reaction that would otherwise produce glutamate. The glutamate dehydrogenase enzyme appears to have a very low affinity for ammonia.

The glutamate generated can also form N-acetylglutamate with acetyl-CoA, catalyzed by a mitochondrial, N-acetyltransferase enzyme called N-acetylglutamate synthase (NAGS). A deficiency in NAGS causes hyperammonemia since N-acetylglutamate is a required allosteric cofactor in the first step of the urea cycle, catalyzed by the enzyme carbamoyl phosphate synthetase I (CPS I), converting ammonia to carbamoyl phosphate.

One can see that nitrogen metabolism in the body, which includes glutamine and glutamate metabolism, the urea cycle, and the balancing of ammonia levels, is complex with many regulated pathways.

While intravenous Ammonul is a standard of care treatment for hyperammonemia, it is often a slow process and infusions may be required for over several days or much longer, while requiring multiple vials of Ammonul. Protein is most often withheld from the patient for 48 hours, to keep ammonia levels from increasing higher, but any longer than 48 hours and the body starts to break down its own proteins which would increase ammonia levels. After achieving normal ammonia levels, protein (or essential amino acid solutions) are gradually reintroduced to prevent sudden rises in ammonia levels. When ammonia levels come down and stay within acceptable levels while receiving protein intake, then Ammonul infusions can be stopped and replaced with oral ammonia scavenger medication. However, Ammonul may be needed again if ammonia levels rebound to hyperammonemia, so there is a transition period.

Ammonul's labeling includes an analysis of over 1,000 hospitalized acute hyperammonemic episodes in over 300 patients with a mean duration of Ammonul treatment of 4.6 days with a standard deviation of 6.45 days, and ranged from 1 to 72 days of Ammonul treatment. About 20% of the patients studied had died, while 80% of patients studied survived their last episode of hyperammonemia.

Some patients are very responsive to Ammonul treatment and high plasma ammonia levels can be halved, e.g., from about 200 micromol per liter down to about 100 micromol per liter, within a matter of 4 hours or so. Other patients are not that responsive to Ammonul and may be recommended as candidates for hemodialysis, such as those patients whose plasma ammonia levels fail to decrease to below 150 micromol per liter or by more than 40% within 4 to 8 hours after receiving Ammonul.

Pharmacokinetic studies in healthy adult volunteers receiving a 1.5 hour priming infusion of Ammonul followed by a 24 hour maintenance infusion of Ammonul demonstrated that benzoate concentrations were undetectable at 14 hours, while phenylacetate was still detected in blood plasma at the end of the infusion. Likewise, according to Ammonul's labeling, the plasma levels of phenylacetate in hyperammonemic patients were higher than benzoate and were present for a longer time following infusion of Ammonul.

The labeling for Ammonul states that there is a difference in the metabolic rates for phenylacetate and benzoate. The formation of hippurate from benzoate (by combining with glycine) occurred more rapidly than that of phenylacetylglutamine from phenylacetate (combining with glutamine), and the rate of elimination for hippurate appeared to be more rapid than that for phenylacetylglutamine. Both benzoate and phenylacetate are said to have saturable, non-linear elimination, with a decrease in clearance with increased dose.

Another explanation is surmised that once the systemic glutamine pool is depleted, the hyperammonemic body is especially slow to make more glutamine, so that Ammonul (i.e., phenylacetate) appears to become quickly inefficient at sequestering glutamine over time. It is hypothesized that the precursor to glutamine, which is the amino acid glutamate, may be in short supply. Another explanation is surmised that the enzyme glycine benzoyltransferase is more efficient at conjugating glycine than the enzyme glutamine phenylacetyltransferase is at conjugating glutamine.

Adverse reactions listed in Ammonul's labeling include: blood and lymphatic system disorders, including coagulopathy, pancytopenia, and thrombocytopenia; cardiac disorders, including atrial rupture, bradycardia, cardiac or cardiopulmonary arrest/failure, cardiogenic shock, cardiomyopathy, and pericardial effusion; blindness; gastrointestinal disorders, including abdominal distension and gastrointestinal hemorrhage; general disorders and administration-site conditions, including asthenia, brain death, chest pain, multiorgan failure, and edema; hepatobiliary disorders, including cholestasis, hepatic artery stenosis, hepatic failure/hepatotoxicity, and jaundice; infections and sepsis or septic shock; injury, poisoning and procedural complications, including brain herniation, subdural hematoma, and overdose; investigations, including blood carbon dioxide changes, blood glucose changes, blood pH increased, cardiac output decreased, pCO₂ changes, and respiratory rate increased; metabolism and nutrition disorders, including: alkalosis, dehydration, fluid overload/retention, hypoglycemia, hyperkalemia, hypernatremia, alkalosis, and tetany; neoplasms that are benign, malignant and unspecified, including hemangioma acquired; nervous system disorders, including areflexia, ataxia, brain infarction, brain hemorrhage, cerebral atrophy, clonus, depressed level of consciousness, encephalopathy, nerve paralysis, intracranial pressure increased, subdural hematoma, and tremor; psychiatric disorders, including acute psychosis, aggression, confusional state, and hallucinations; renal and urinary disorders, including anuria, renal failure, and urinary retention; respiratory, thoracic and mediastinal disorders, including acute respiratory distress syndrome, dyspnea, hypercapnia, hyperventilation, Kussmaul respiration, pneumonia aspiration, pneumothorax, pulmonary hemorrhage, pulmonary edema, respiratory acidosis or alkalosis, and respiratory arrest/failure; skin and subcutaneous tissue disorders, including: alopecia, blister, pruritis generalized, rash, and urticaria; and vascular disorders, including flushing, hemorrhage, hypertension, and phlebothrombosis/thrombosis. Additional adverse reactions include nausea, vomiting, anemia, and hypotension.

Another risk of prolonged, multiday maintenance of phenylacetate infusions is potential neurotoxicity associated with phenylacetate that was suggested by Ammonul's labeling in a group of cancer patients, which includes somnolence, fatigue, and lightheadedness, as well as, headaches and exacerbation of existing neuropathy. Another risk with Ammonul is potential for hyperventilation and metabolic acidosis. There is also a tendency for decreased potassium levels.

After several hours to several days or more when ammonia levels have been significantly lowered after a hyperammonemia crisis, patients are transitioned to oral ammonia scavengers generally instead of intravenous ones. Often, these patients (other than newborns) had been taking oral ammonia scavengers for a long time before their hyperammonemia crisis began; in such cases, the hyperammonemia crisis represents a breakthrough case whereby oral ammonia scavengers and dietary management of nitrogen failed to prevent the incident. The present disclosure provides improved methods and oral preparations and formulations to prevent such breakthrough incidents of hyperammonemia from occurring, as well as, improved methods and intravenous preparations and formulations to help clear hyperammonemia faster if it should still occur. The term preparation or pharmaceutical preparation includes drug(s) or medication(s) intended for human or veterinary use, usually in their finished dosage form, and can include single ingredient preparations, as well as, formulations with at least one active ingredient and at least one other ingredient.

Oral ammonia scavenger drugs; also called oral nitrogen-binding agents or oral nitrogen scavengers herein; are FDA approved drugs indicated as adjunctive therapy to standard of care, which includes dietary management, for the chronic management of adult and pediatric patients with urea cycle disorders (UCDs), involving deficiencies of carbamylphosphate synthetase (CPS), ornithine transcarbamylase (OTC) or argininosuccinic acid synthetase (AS). An alternative indication is for use as a nitrogen-binding agent for chronic management of patients with urea cycle disorders (UCDs) who cannot be managed by dietary protein restriction and/or amino acid supplementation alone; and must be used with dietary protein restriction and, in some cases, dietary supplements (e.g., essential amino acids, arginine, citrulline, protein-free calorie supplements).

Importantly, episodes of acute hyperammonemia may occur in patients while on these oral ammonia scavenger drugs, and such oral ammonia scavengers are not indicated for the treatment of acute hyperammonemia in patients with UCDs because more rapidly acting (intravenous) interventions are essential to reduce plasma ammonia levels.

Moreover, these oral ammonia scavengers are for patients with urea cycle disorders, and are not currently indicated for patients having liver disease and or liver dysfunction, the other causes of elevated ammonia levels in the blood. Even intravenous ammonia scavengers, such as Ammonul, are only indicated for use in patients with urea cycle enzyme deficiencies, and not patients with liver disease or liver impairment. For patients with liver disease, new intravenous and oral ammonia scavengers are needed to treat or prevent hepatic encephalopathy or its reoccurrence. The present disclosure provides methods and oral formulations and intravenous formulations for reducing nitrogen (ammonia) levels in patients with liver disease (e.g., cirrhosis, hepatic steatosis) and or liver impairment, and not just in patients with urea cycle enzyme deficiencies.

Oral ammonia scavengers once included oral formulations of phenylacetate. However, oral phenylacetate had a very displeasing odor and taste, and has since been replaced with oral phenylbutyrate, which is a prodrug of phenylacetate which gets beta oxidized into phenylacetate. Oral drugs containing or providing phenylbutyrate come in various forms. Phenylbutyrate also has a foul taste though. Buphenyl® is an approved drug product that contains sodium phenylbutyrate in oral powder form, and its foul taste makes it difficult for pediatric patients to take.

Manufacturers have found ways to help taste-mask phenylbutyrate. Olpruva® is an approved drug supplied in a kit of packets of taste-masked, sodium phenylbutyrate pellets that are mixed into a glass of water to form an oral suspension before use. The Olpruva kit comes with a suspending agent or mix-aid, a separate packet of corn starch for suspension. The mixing, suspending, and administration of Olpruva several times a day, such as three to six doses, can become very cumbersome, leading to noncompliance. Additionally, the formulation of Olpruva contains talc, which in other types of products has been suspected to contain asbestos and or be carcinogenic. Olpruva can only be orally swallowed as it is not permitted for use with gastrostomy or nasogastric tubes, otherwise known as feeding tubes. Olpruva also has a very high price.

Pheburane® is an approved drug product of taste-masked sodium phenylbutyrate pellets supplied in a bottle and used with a calibrated measuring spoon. Pheburane pellets must be either sprinkled on applesauce and swallowed that way, or swallowed with a drink. Unlike Olpruva, Pheburane cannot be mixed into a drink because the taste-masked coating of the pellets will dissolve, and then the product becomes unpalatable. The taste-masked coating may even dissolve in the mouth if not swallowed right away. This too may pose challenging when dosing in very young pediatric patients. Like Olpruva, Pheburane can only be orally swallowed as it is not permitted for use with gastrostomy or nasogastric tubes, otherwise known as feeding tubes. The three to six doses required per day can also be cumbersome, leading to noncompliance.

An alternative product to Buphenyl, Olpruva, and Pheburane is Ravicti®. Instead of sodium phenylbutyrate as an active ingredient, which requires taste masking and results in a solid dosage form (e.g., pellets), Ravicti uses glycerol phenylbutyrate [chemical name: benzenebutanoic acid, 1′,1″-(1,2,3-propanetriyl) ester and also called 2,3-bis(4-phenylbutanoyloxy)propyl 4-phenylbutanoate] as its active ingredient and only ingredient. Ravicti is considered an oral liquid, and a new dry oral syringe is required to measure and administer each dosage. The benefit of Ravicti is that it can be used with patients having gastrostomy or nasogastric tubes. Ravicti is a relatively expensive product too, but the market seems to appreciate its easier dosage and administration. Ravicti does not require taste-masking as glycerol phenylbutyrate is much more palatable than sodium phenylbutyrate.

Glycerol phenylbutyrate, as Ravicti's product insert describes, is a triglyceride containing three molecules of phenylbutyrate bound as esters to a glycerol backbone. When ingested, exocrine pancreatic lipases in the small intestine hydrolyze and separate glycerol phenylbutyrate into glycerol and three molecules of phenylbutyrate. The phenylbutyrate molecules are then absorbed into the circulation and beta-oxidized into phenylacetate. Then, in the liver, the phenylacetate conjugates with glutamine to form phenylacetylglutamine for subsequent elimination in the urine. Note however, patients with low or absent pancreatic enzymes or intestinal disease resulting in fat malabsorption may result in reduced or absent digestion of Ravicti and or absorption of phenylbutyrate and reduced control of plasma ammonia.

Generally, Ravicti seems like a great product. However, there is believed to be a major problem with Ravicti for many, if not all, patients, which is not discussed in Ravicti's product insert. The recommended maximum total daily dosage of Ravicti is 17.5 mL (19 grams). When Ravicti is ingested, much of that large, ingested amount yields glycerol, which is absorbed. Glycerol is simple triol compound consisting of a three-carbon atom chain with a hydroxyl group on each carbon. The ingestion of glycerol can result in increased levels of triacylglycerol (triglycerides) in the blood, along with increased total cholesterol and an increased diameter of adipocytes (fat cells). Triglycerides are an ester derived from glycerol and three fatty acids; each fatty acid forms an ester with each hydroxyl group of glycerol. Triglycerides are the main constituents of body fat. Fats are stored in adipose tissue. But fats are also transported around the body via lipoproteins, such as low-density lipoprotein (LDL) particles, in the blood. Sometimes these LDL particles become oxidized and form plaques in blood vessel walls to cause atherosclerosis. So once glycerol is absorbed after taking Ravicti, this glycerol is believed to form fatty triglycerides and lead to more cholesterol and fat in the body. So while Ravicti is benefiting patients with urea cycle disorders to help manage their excess nitrogen, Ravicti might actually be harming these patients in other ways, from a cardiovascular and metabolic perspective. Ravicti is meant for chronic use. High amounts of triglycerides, cholesterol, and fat, caused or exacerbated by Ravicti, can eventually lead to heart disease, cardiovascular disease, and or atherosclerosis. It is believed that Ravicti may cause dyslipidemia. Additionally, some patients may have glycerol intolerance for which ingesting or absorbing glycerol can cause serious adverse conditions and may require hospitalization. Ravicti should be avoided in a patients with glycerol intolerance.

### EXAMPLE EMBODIMENTS

The present disclosure includes methods, compositions, and formulations to help caregivers and their patients with urea cycle disorders and or liver disease to manage nitrogen and or ammonia levels, and help reduce or prevent the occurrence of hyperammonemia, by providing a palatable, preferably liquid, oral nitrogen-binding agent that preferably does not increase glycerol absorption and or glycerol levels, and preferably does not appreciably increase at least one of triglyceride levels, cholesterol levels, fat production, and or risk of cardiovascular disease. The present disclosure also includes compounds of new active pharmaceutical ingredients (APIs) along with their creation and methods of manufacture and uses, as well as, pharmaceutical preparations including one or more of these active pharmaceutical ingredients and their methods of use.

In the following embodiments, the terms nitrogen-binding agent is analogous and or equivalent to the terms nitrogen-scavenging agent or ammonia-scavenging agent or ammonium-scavenging agent. These terms generally refer to the removal of nitrogenous compounds from the body. For example, these agents may be involved in the removal of nitrogenous amino acids, such as glutamine or glycine.

A first embodiment of the present disclosure is a nitrogen-binding agent comprising an at least one ester of phenylbutyrate attached to the hydroxyl group of an alcohol molecule, wherein the alcohol molecule is not the triol glycerol, and therefore, does not consist of a glycerol backbone. In many embodiments, this nitrogen-binding agent is administered orally or by feeding tube, and in other embodiments may be administered intravenously.

A second embodiment of the present disclosure is a nitrogen-binding agent comprising two esters of phenylbutyrate attached to the hydroxyl groups of an alcohol molecule, wherein the alcohol molecule is a glycol (or diol), and not the triol glycerol, and therefore, does not consist of a glycerol backbone. In many embodiments, this nitrogen-binding agent is administered orally or by feeding tube, and in other embodiments may be administered intravenously. In many embodiments, a palatable, liquid nitrogen-binding agent is preferred. In some embodiments, the diol may be a sugar alcohol.

Examples of second embodiments include the nitrogen-binding agent resulting from the esterification of phenylbutyrate with diols, including, but not limited to, at least one of propylene glycol; 1,3-propanediol; 1,3-butanediol; or a combination thereof. These diols are liquids at room temperature and have a very low melting point. When two molecules of phenylbutyrate are esterified to the sugar alcohol called propylene glycol, the resulting nitrogen-binding agent is propane-1,2-diyl bis(4-phenylbutanoate). When two molecules of phenylbutyrate are esterified to the diol called 1,3-propanediol, the resulting nitrogen-binding agent is propane-1,3-diyl bis(4-phenylbutanoate). When two molecules of phenylbutyrate are esterified to the diol called 1,3-butanediol, the resulting nitrogen-binding agent is butane-1,3-diyl bis(4-phenylbutanoate).

Propylene glycol is a liquid sugar alcohol used in foods and pharmaceuticals, and is safe in large amounts. As a sugar alcohol, propylene glycol has a somewhat sweet taste. The diol called 1,3-propanediol is derived from corn sugar and is said to be about 60% as sweet as sucrose, with a pleasing, candy-like smell. This diol is also safe to ingest. The diol called 1,3-butanediol is bittersweet, meaning it has a sweet flavor with a bitter aftertaste, and is a food additive and is generally regarded as safe (GRAS). The safety of these diols is important as once the nitrogen-binding agent is ingested and hydrolyzed to release phenylbutyrate molecules in the intestines for absorption, these diols are also absorbed. For instance, propylene glycol has a rapid absorption.

A third embodiment of the present disclosure is a nitrogen-binding agent comprising more than two esters of phenylbutyrate attached to the hydroxyl groups of an alcohol molecule, wherein the alcohol molecule is not the triol glycerol, and therefore, does not consist of a glycerol backbone. An example of this third embodiment includes the nitrogen-binding agent resulting from the esterification of four molecules of phenylbutyrate with the four hydroxyl groups of the sweet sugar alcohol, erythritol. The resulting nitrogen-binding agent, erythrityl-tetra-(4-phenylbutanoate) is believed to have great taste-masking properties and may provide for oral pellets, powders, capsules, and or tablets. When hydrolyzed in the intestines, the sugar alcohol erythritol is released, along with four molecules of phenylbutyrate for absorption. In many embodiments, this nitrogen-binding agent is administered orally, and in other embodiments may be administered intravenously when dissolved in a solvent (e.g., water for injection or saline for injection).

In further embodiments, the nitrogen-binding agent according to the present disclosure is co-administered with ornithine.

In still further embodiments, ornithine is included in the formulation with the nitrogen-binding agent according to the present disclosure.

Ornithine is a non-proteinogenic amino acid generally known for its role in the urea cycle wherein arginine produces the products urea and ornithine. Ornithine contains two amino groups. Ornithine as an ingredient can lead to more glutamate production in the body, which in turn leads to more glutamine production in the body. Ornithine delta-aminotransferase is an enzyme that transfers the delta-amino group from ornithine to alpha-ketoglutarate to form glutamate-5-semialdehye and glutamate. The glutamate-5-semialdehyde can itself form a molecule of glutamate from the enzyme glutamate-5-semialdehyde dehydrogenase. Therefore, each molecule of ornithine can yield two molecules of glutamate. These two molecules of glutamate can each form glutamine while each sequestering an ammonium ion with the enzyme glutamine synthetase. Each glutamine molecule formed can then conjugate with phenylacetate, by the enzyme called glutamine phenylacetyltransferase, to form phenylacetylglutamine that is subsequently eliminated in the urine. In this manner, ornithine can increase the throughput of this nitrogen-scavenging and elimination pathway when used with the nitrogen-binding agent according to the present disclosure; the nitrogen-binding agent serving as a source of phenylbutyrate, the prodrug of phenylacetate.

In further embodiments, the nitrogen-binding agent according to the present disclosure is co-administered with ornithine benzoate.

In still further embodiments, ornithine benzoate is included in the formulation with the nitrogen-binding agent according to the present disclosure.

Ornithine benzoate as an active pharmaceutical ingredient has two advantages. Ornithine, as discussed above, enhances the throughput of the nitrogen-scavenging pathway of phenylacetate by producing more glutamine for removal. Meanwhile, benzoate conjugates with glycine to form hippurate by the enzyme glycine benzoyltransferase, and the resulting hippurate is eliminated in the urine. Each glycine removed is equivalent to one nitrogen unit removed.

The phenylbutyrate esters formed from glycols (or diols) according to second embodiments, each having two such 4-phenylbutanoate groups, are found to be ideal for use with ornithine or ornithine benzoate, when used in a one-to-one (1:1) ratio. The phenylbutyrate esters formed from glycols (or diols), such as propane-1,2-diyl bis(4-phenylbutanoate); propane-1,3-diyl bis(4-phenylbutanoate); and or butane-1,3-diyl bis(4-phenylbutanoate); each release two molecules of phenylbutyrate when hydrolyzed, subsequently forming two molecules of phenylacetate. These two molecules of phenylacetate can conjugate to two molecules of glutamine for removal as two molecules of phenylacetylglutamine. As discussed above, each molecule of ornithine can result in the formation of two molecules of glutamine. Therefore, providing a nitrogen-binding agent having two 4-phenylbutanoate groups, according to second embodiment(s), in a one-to-one (1:1) ratio with ornithine or ornithine benzoate provides a stoichiometrically balanced elimination of two glutamines. In other words, the ornithine creates two glutamine molecules and the two phenylbutyrate molecules help remove these two glutamine molecules from the urine.

In contrast, providing ornithine with the nitrogen-binding agent glycerol phenylbutyrate of Ravicti would not have this stoichiometric balance as glycerol phenylbutyrate yields three molecules of phenylbutyrate when hydrolyzed.

A fourth embodiment of the present disclosure is a nitrogen-binding agent comprising a PEGylated form of phenylbutyrate, or PEGylated phenylbutyrate. PEGylation refers to the attachment of polyethylene glycol polymer chains to molecules, and in this instance, the attachment to phenylbutyrate or a derivative thereof. Polyethylene glycol (PEG) is a long carbon chain with a terminal hydroxyl group on both ends. There also exist branched and star forms of PEG, with multiple PEG chains emanating from a central core group. Other forms of PEG are also possible. PEG has various lengths and molecular weights. PEG is often used as an excipient in pharmaceutical products. For most, PEG is safe and inert. In some fourth embodiments, the nitrogen-binding agent comprises polyethylene glycol having an ester of phenylbutyrate on both terminal ends. In other fourth embodiments, the nitrogen-binding agent comprises polyethylene glycol having an ester of phenylbutyrate on one terminal end, while the other terminal end is protected and or attached to a different molecule. In still further fourth embodiments, the nitrogen-binding agent comprises a mixture of polyethylene glycol molecules, some PEGs having a phenylbutyrate ester on only one end and other PEGs having phenylbutyrate esters on both ends. Some of these embodiments utilize PEG of equal length, and other embodiments utilize PEG of variable lengths and molecular weights.

Certain embodiments comprise methods of manufacturing said first, second, third, and/or fourth embodiments. Such methods of manufacturing comprise, but not limited to, esterification reactions, along with methods of purifying, compounding, and filling these phenylbutyrate esters, including their liquid, oral nitrogen-binding agent forms. Additional methods of formulating these phenylbutyrate esters with other active and inactive ingredients also reflect certain embodiments, and this disclosure is not meant to be limiting.

In further embodiments, a polymer other than PEG may be used with and or attached to phenylbutyrate or derivatives thereof.

In oral formulations of or containing PEGylated phenylbutyrate, PEGylation can taste-mask phenylbutyrate molecules. In some embodiments, PEGylation can differentially slow down the hydrolyzation of phenylbutyrate esters (i.e., its release) in the intestines.

In oral formulations of or containing PEGylated ornithine, in some embodiments, PEGylation can differentially slow down the absorption of ornithine.

In intravenous formulations of PEGylated phenylbutyrate, in some embodiments, PEGylation can differentially slow down the beta oxidation of phenylbutyrate into phenylacetate.

Still further embodiments are described in the present disclosure.

In oral formulations of or containing PEGylated benzoate, in some embodiments, PEGylation can differentially slow down the absorption of benzoate.

In intravenous formulations of or containing PEGylated phenylacetate, in some embodiments, PEGylation can differentially slow down the conjugation of phenylacetate with glutamine.

In intravenous formulations of or containing PEGylated ornithine, in some embodiments, PEGylation can differentially slow down the conversion of ornithine into glutamate.

In intravenous formulations of or containing PEGylated benzoate, in some embodiments, PEGylation can differentially slow down the conjugation of benzoate with glycine.

These above embodiments and examples are not meant to be limiting.

Differential release and or slow or delayed release formats of the oral nitrogen-binding agent(s), PEGylated or otherwise, can allow for a reduction in the number of doses per day. This is important as products such as Pheburane and Olpruva may be dosed six times per day, while Ravicti may be dosed many times per day as well. A reduced number of doses per day can increase patient compliance, which in turn, can reduce breakthrough hyperammonemia events.

Other embodiments are envisioned in which intravenous formulations including phenylacetate and or phenylbutyrate, and or esters or amides or derivatives thereof, are also differentially released or exposed to allow for extended pharmacokinetic profiles from a bolus intravenous dose or rapid intravenous infusion. This may also include phenylacetate and or phenylbutyrate molecules or esters or amides or other derivatives surrounded by a slow release molecular cage, whether with PEG or other types of polymers. Liposomes and other vesicle formats are also possible for this purpose.

The dosage of phenylacetate (e.g., intravenous phenylacetate) or phenylbutyrate (e.g., oral phenylbutyrate or esters thereof) is limited by the potential for toxicity of phenylacetate. For example, at least some of the side effects or adverse reactions to Ammonul and oral drugs of phenylbutyrate or Ravicti are believed due to elevated or residual levels of phenylacetate in the blood after patients receive these medications.

The enzyme glutamine phenylacetyltransferase is responsible for the conjugation of glutamine and phenylacetate to form phenylacetylglutamine. This enzyme or process may have lower efficiency and or become saturated as levels of phenylacetate increase in the blood plasma, over levels of phenylacetylglutamine, with increasing dose and or decreased liver function.

A biotech solution of providing a recombinant enzyme formulation of glutamine phenylacetyltransferase, and or alternatively a biotech solution of providing a DNA- and or RNA-based expression system of the enzyme glutamine phenylacetyltransferase, to patients may not only alleviate some of the toxicity and side effects of excess phenylacetate in the blood, but can allow for much higher doses of intravenous phenylacetate and or oral phenylbutyrate (e.g., phenylbutyrate esters or amides). This can greatly enhance the overall throughput of glutamine (nitrogen) elimination and faster clearance of hyperammonemia events. Such a biotech solution in combination with one or more of the nitrogen-binding agent embodiments above (e.g., first embodiment, second embodiment, third embodiment, fourth embodiment, or a combination thereof) can prevent hyperammonemia from ever occurring and or quickly alleviate hyperammonemia should it occur. Such a combination should help negate the need for hemodialysis should hyperammonemia occur and or reduce the time spent by the patient in the intensive care unit. Still further, this biotech solution may alternatively or additionally comprise a recombinant enzyme formulation of glycine benzoyltransferase, and or alternatively a biotech solution of providing a DNA- and or RNA-based expression system of the enzyme glycine benzoyltransferase; which would enhance the conjugation of glycine and benzoate into hippurate for elimination.

Enzyme expression systems are DNA- and or RNA-based expression systems coding for one or more of these enzymes. An example may include a DNA plasmid coding for one or more of these enzymes. Another example may include a viral vector (e.g., an adenovirus vector) containing the coding for one or more of these enzymes. Another example may include something similar to an RNA vaccine coding for one or more of these enzymes. These examples and ways of expressing these enzymes in the body are not meant to be limiting. The technology behind gene therapies may also be included to help increase expression of one or more of these enzymes. Expression of promoters and or transcription factors of the genes coding for one or more of these enzymes are also encompassed by this disclosure.

Methods of increasing or enhancing the oral bioavailability of at least one of glutamate, glutamine, ornithine, benzoate, phenylbutyrate, phenylbutyrate ester, phenylbutyrate amide, recombinant enzyme of glycine benzoyltransferase, recombinant enzyme of glutamine phenylacetyltransferase, derivative or a combination thereof are included for oral pharmaceutical formulations, and may permit bypassing of first-pass metabolism.

Certain embodiments comprise methods, formulations, and applications of managing blood nitrogen levels, which include new and improved pharmacologic treatments and or biotechnology treatments.

The present disclosure includes methods of treating, preventing, or reducing hyperammonemia, which include new and improved pharmacologic treatments and or biotechnology treatments. The present disclosure includes methods, compositions, and formulations to help caregivers and their patients with urea cycle disorders and or liver disease to manage nitrogen and or ammonia levels, and help reduce or prevent the occurrence of hyperammonemia, by providing a palatable, preferably liquid, oral nitrogen-binding agent that preferably does not increase glycerol absorption and or glycerol levels, and preferably does not appreciably increase at least one of triglyceride levels, cholesterol levels, fat production, and or risk of cardiovascular disease. Many of the embodiments of the disclosure revolve around providing better and or safer phenylbutyrate esters for oral administration.

Other aspects of the present disclosure include increasing glutamine formation by providing oral ornithine and or injectable ornithine, or alternatively, oral ornithine benzoate and or injectable ornithine benzoate.

Other aspects of the present disclosure include increasing glutamine formation by its immediate precursor(s) glutamate and or glutamyl-phosphate for injection, and or providing the enzyme glutamine synthetase for injection.

Such methods of the present disclosure can also increase the efficiency and or efficacy of each infusion of sodium phenylacetate and or sodium benzoate injection so that ammonia can be cleared from the body faster.

The present disclosure also includes methods that can decrease one or more of the side effects/adverse reactions of each infusion of sodium phenylacetate and or sodium benzoate injection, and or decrease one or more of the side effects/adverse reactions of the overall treatment regimen, especially if less days and a smaller number of infusions of sodium phenylacetate and or sodium benzoate injection are required with these methods. The reduction or elimination of one or more of the above side effects or adverse reactions by the present disclosure and its methods would be very desirable when treating hyperammonemia patients.

Other forms of the molecules mentioned in the embodiments and or examples above may be apparent, and this disclosure is intended to encompass these additional forms, whether other prodrugs, salts, derivatives, enantiomers, or combinations thereof. Moreover, cell membrane permeable forms of any of the molecules mentioned in the embodiments and or examples above may be apparent, and this may include adding other functional groups to these molecules, or by using vesicle-like, microspheres of one or more of these molecules. For example, dimethyl glutamate may be more membrane permeable than glutamate. For example, polyglutamate microspheres; e.g., sodium polyglutamate microspheres; which are vesicle-like spheres made from glutamic acid molecules linked together through amide bonds. These polyglutamate microspheres can be transported through the blood, and can contain, and be used to deliver, additional pharmaceutical ingredient(s), such as a nitrogen-binding agent. Polyglutamate can be broken down into glutamate molecules by protease enzymes, such as cathepsin present in lysosomes. Polyglutamate microspheres may also be considered as prodrugs of glutamate.

Formulations, including oral formulations, of any of the above embodiments and or examples may optionally include an at least excipient ingredient. Pharmaceutically acceptable excipients or solvents may be included in the formulation, such as acids or bases to adjust pH, or salts to adjust osmolarity, preservatives, stabilizers, or even other solvents, carriers, and polymers. Example excipient ingredients may include salts like sodium chloride and potassium bicarbonate, detergents and or chelators such as EDTA, certain polymers, pH adjustors (e.g., acids or bases), liposomes, preservatives, tonicity agents, solvents or water. Pharmaceutically acceptable excipients are preferred. These classes of excipients and or excipient examples are not meant to be limiting to one skilled in the art. Although preferred, oral preparations may not need to be necessarily sterile, but at the very least, to have low or negligible endotoxin levels. It is also desirable for the formulation to have good stability, low impurities, and a shelf life of at least six months, and preferably a shelf life of at least one year or more.

Injectable formulations of any of the above embodiments and or examples may optionally include an at least excipient ingredient. Pharmaceutically acceptable excipients or solvents may be included in the formulation, such as acids or bases to adjust pH, or salts to adjust osmolarity, preservatives, stabilizers, or even other solvents, carriers, and polymers. Example excipient ingredients may include salts like sodium chloride and potassium bicarbonate, detergents and or chelators such as EDTA, certain polymers, pH adjustors (e.g., acids or bases), liposomes, preservatives, tonicity agents, solvents or water. Pharmaceutically acceptable excipients are preferred. These classes of excipients and or excipient examples are not meant to be limiting to one skilled in the art. It is desirable for the injectable formulation to be sterile and have low or negligible endotoxin levels. It is also desirable for the formulation to have good stability, low impurities, and a shelf life of at least six months, and preferably a shelf life of at least one year or more.

In some embodiments, an at least one injectable pharmaceutical formulation of ornithine benzoate and phenylacetate, or a salt, prodrug (e.g., phenylbutyrate or phenylbutyrate ester), derivative, or combination thereof is in an aqueous solution. In the simplest embodiment, the at least one injectable pharmaceutical formulation is in water or saline for injection, with water as a solvent and or saline salts, and optionally dextrose, as the only excipient ingredient(s). However, the injectable solution preferably contains pH adjusters, such as sodium hydroxide and or hydrochloric acid. The concentration of the ornithine benzoate and phenylacetate, or a salt, prodrug (e.g., phenylbutyrate or phenylbutyrate ester), derivative, or combination thereof in the formulation can each range from 0.1 mg/mL to over 50 mg/mL, and in many cases approach 100 mg/mL or more. By adjusting the pH and or using solubility enhancers, some embodiments have a concentration that can approach, reach, or exceed 101 mg/mL. In some embodiments, different concentrations are used at different times and or for different infusions. In other embodiments, the concentration is titrated based on blood or plasma measurements, such as of ammonia or ammonium and other metabolites or biochemical markers. Alternatively, an injectable suspension embodiment or an implantable formulation embodiment may be desirable for maximizing potency and concentration, or providing a means of a supersaturated form of ornithine benzoate and phenylacetate, or a salt, prodrug (e.g., phenylbutyrate or phenylbutyrate ester or amide), derivative, or combination thereof.

In some embodiments, the concentration of the ornithine benzoate in the formulation can be less than, equivalent to, or greater than the concentration of phenylacetate, or a salt, prodrug (e.g., phenylbutyrate or phenylbutyrate ester or amide), derivative, or combination thereof being infused.

In some embodiments, the concentration of the ornithine benzoate in the formulation can be less than, equivalent to, or greater than the concentration of phenylacetate, or a salt, prodrug (e.g., phenylbutyrate or phenylbutyrate ester or amide), derivative, or combination thereof included in the formulation.

In some embodiments, an at least one pharmaceutical formulation of an at least one injectable pharmaceutical formulation of ornithine benzoate and phenylacetate, or a salt, prodrug (e.g., phenylbutyrate or phenylbutyrate ester or amide), derivative, or combination thereof further comprises at least one excipient ingredient selected from solvents, tonicity agents, chelators, buffers, pH adjusting agents, solubility enhancing agents, preservatives, or a combination thereof. Water is an aqueous solvent. Various other solvents exist and can be employed as pharmaceutically acceptable solvents including organic and inorganic solvents, alcohols, e.g., ethanol, butanol, ketones, e.g., acetone, ethers, e.g., cyclopentyl methyl ether, oils, fatty acids and carboxylic acids, e.g., octanoic acid, solvent disulfides, and eutectic solvents. These examples are not meant to be limiting. Tonicity agents and salts can include sodium chloride and potassium chloride, and possibly sugars. These examples are not meant to be limiting. Chelators can include ethylenediaminetetraacetic acid (EDTA). These examples are not meant to be limiting. Buffers can include citric acid buffers, acetic acid buffers and sodium phosphate buffers. These examples are not meant to be limiting. Agents that adjust pH (pH adjusting agents) can include sodium hydroxide, acetic acid, hydrochloric acid, calcium carbonate, and gluconic acid. These examples are not meant to be limiting. Solubility enhancing agents can include polymers and liposomes. These examples are not meant to be limiting. Preservatives can include sodium benzoate (such as when sodium benzoate is not included as an active ingredient), benzyl alcohol, metabisulfites, and chlorobutanol. These examples are not meant to be limiting. Various combinations of two or more of any of these excipients is possible and can be employed.

In some embodiments, an at least one pharmaceutical formulation including an at least one phenylbutyrate ester or phenylbutyrate amide further comprises at least one excipient ingredient selected from solvents, tonicity agents, chelators, buffers, pH adjusting agents, solubility enhancing agents, preservatives, or a combination thereof. Water is an aqueous solvent. Various other solvents exist and can be employed as pharmaceutically acceptable solvents including organic and inorganic solvents, alcohols, e.g., ethanol, butanol, ketones, e.g., acetone, ethers, e.g., cyclopentyl methyl ether, oils, fatty acids and carboxylic acids, e.g., octanoic acid, solvent disulfides, and eutectic solvents. These examples are not meant to be limiting. Tonicity agents and salts can include sodium chloride and potassium chloride, and possibly sugars. These examples are not meant to be limiting. Chelators can include ethylenediaminetetraacetic acid (EDTA). These examples are not meant to be limiting. Buffers can include citric acid buffers, acetic acid buffers and sodium phosphate buffers. These examples are not meant to be limiting. Agents that adjust pH (pH adjusting agents) can include sodium hydroxide, acetic acid, hydrochloric acid, calcium carbonate, and gluconic acid. These examples are not meant to be limiting. Solubility enhancing agents can include polymers and liposomes. These examples are not meant to be limiting. Preservatives can include sodium benzoate (such as when sodium benzoate is not included as an active ingredient), benzyl alcohol, metabisulfites, and chlorobutanol. These examples are not meant to be limiting. Various combinations of two or more of any of these excipients is possible and can be employed.

In some embodiments, only a single active ingredient is included in an at least one pharmaceutical formulation.

In some embodiments, only a single active ingredient is included in an at least one pharmaceutical formulation and that pharmaceutical formulation is to be used in combination or co-administered with a pharmaceutical ingredient containing a different active ingredient.

In some embodiments, two active ingredients are included in an at least one pharmaceutical formulation.

In some embodiments, three or more active ingredients are included in an at least one pharmaceutical formulation.

One or more additional active ingredients may come from different classes of drugs, such as other drugs that treat urea cycle disorders; by way of example, including citrulline and or arginine for patients with CPS or OTC deficiency; and or carglumic acid for patients with NAGS deficiency.

Methods of the present disclosure optionally include steps to measure levels of one or more of ammonia, ammonium ions, ornithine, alpha-ketoglutarate, glutamate, glutamine, urea, benzoate, phenylacetate, phenylacetylglutamine, hippurate, ATP, and or metabolites thereof, as well as other biochemical markers, in whole blood, blood plasma, blood cells, and or tissue samples; before treatment, during treatment, after treatment, or a combination thereof with one or more pharmaceutical preparations or formulations of one or more of the above embodiments of this disclosure. For example, whole blood can be tested for total glutamine levels and or even phenylacetylglutamine levels with liquid chromatography/tandem mass spectrometry and or kinetic spectrophotometry, before, during, and or after treatment with pharmaceutical formulations of one or more of the above embodiments of this disclosure. Methods of the present disclosure optionally include steps to gene sequence or detect genetic mutations in, and or measure levels of, and or measure levels of activity of, one or more enzymes involved in and or related to the metabolism of nitrogen, or a combination above. Methods of the present disclosure also include steps to control and or titrate levels of intracellular glutamine and or its metabolites, up or down, based on repeated assay measurements.

Methods of the present disclosure include administering an at least one pharmaceutical preparation or formulation of an at least one nitrogen-binding agent according to the present disclosure for the treatment of hyperammonemia. (For injectable preparations, the administering of an at least one pharmaceutical preparation or formulation of an at least one nitrogen-binding agent according to the present disclosure is selected from intravenous bolus injection, intravenous bolus infusion, continuous intravenous infusion, subcutaneous injection, intramuscular injection, intrathecal injection, intraosseous injection, or implantation.) Methods include repeat dosing spaced at least one hour apart, and in other embodiments multiple hours apart, and in still other embodiments, spaced one or more days apart.

Methods of the present disclosure include administering an at least second and or third dose, or more, even many multiple doses, of an at least one pharmaceutical preparation or formulation of an at least one nitrogen-binding agent according to the present disclosure for the treatment of hyperammonemia. (For injectable preparations, the administering of an at least second and or third dose, or more, even many multiple doses, of an at least one pharmaceutical preparation or formulation of an at least one nitrogen-binding agent according to the present disclosure for the treatment of hyperammonemia is selected from intravenous bolus injection, intravenous bolus infusion, continuous intravenous infusion, subcutaneous injection, intramuscular injection, intrathecal injection, intraosseous injection, or implantation.) The administering of an at least second and or third dose, or more, even many multiple doses, of an at least one pharmaceutical preparation or formulation of an at least one nitrogen-binding agent according to the present disclosure for the treatment of hyperammonemia is performed in some embodiments within a year, in other embodiments within a month, in other embodiments within a week, in other embodiments within a day, and in other embodiments within an hour, of the first dose. These methods of repeat dosing are not meant to be limited by these examples and other dosing regimens are possible within the scope of this disclosure.

In some embodiments, an at least one injectable pharmaceutical formulation of an at least one nitrogen-binding agent according to the present disclosure is contained in a single-dose ampoule or vial; multi-dose ampoule, vial, or bottle; or intravenous bag, infusion pump, or a prefilled syringe, cartridge, wearable injector, pen injector, or autoinjector.

In some embodiments, an injectable formulation is a liquid. In some embodiments, an injectable formulation is a gel or semi-solid. In still other embodiments, an injectable formulation is in a concentrated and or dry form and or lyophilized powder form requiring dilution, reconstitution, or dissolution in a solvent (e.g., water or saline for injection) prior to injection.

Some embodiments include an at least one injectable pharmaceutical formulation of an at least one nitrogen-binding agent according to the present disclosure, and optionally ornithine and or ornithine benzoate. Such embodiments include methods of self-injection, and include autoinjectors and or pen injectors and or wearable infusion pump devices and or wearable patch injector devices containing an at least one injectable pharmaceutical formulation of an at least one nitrogen-binding agent according to the present disclosure, and optionally ornithine and or ornithine benzoate, including for use with oral forms of phenylbutyrate to enhance ammonia clearance by increasing the production of glutamine from glutamate, while scavenging extra ammonia from the body. For the management of urea cycle disorders, the administering of the at least one injectable pharmaceutical formulation of an at least one nitrogen-binding agent according to the present disclosure, and optionally ornithine and or ornithine benzoate, is performed in some embodiments at least once per year, in other embodiments at least once per month, in other embodiments at least once per week, in other embodiments at least once per day, and in other embodiments at least once per hour; and optionally while in combination with oral phenylbutyrate. In still other embodiments, the at least one injectable pharmaceutical formulation of an at least one nitrogen-binding agent according to the present disclosure, and optionally ornithine and or ornithine benzoate, also optionally includes phenylacetate (e.g., sodium phenylacetate).

In some embodiments the autoinjector containing an at least one injectable pharmaceutical formulation including of an at least one nitrogen-binding agent according to the present disclosure, and optionally ornithine and or ornithine benzoate, is powered by a spring mechanism. In other embodiments, the autoinjector is powered by compressed gas. In other embodiments, the liquid is contained in a cartridge, while in other embodiments the liquid is contained in a prefilled syringe. In other embodiments a prefilled syringe is administered manually. In yet still other embodiments, the prefilled syringe is administered with the aid of a syringe assist device that the prefilled syringe fits into, also known as a manual assist device. In some embodiments the injector contains a needle. In some embodiments, microneedles may be used. In other embodiments the injector is needle-free and penetrates the skin with compressed gas or air.

In some embodiments, the formulation is delivered by a reusable pen or reusable autoinjector device associated with replaceable containers containing of an at least one nitrogen-binding agent according to the present disclosure, and optionally ornithine and or ornithine benzoate, and optionally phenylacetate.

Some embodiments include administration with a portable infusion pump for prolonged subcutaneous, intramuscular, and or intravenous administration.

Other methods of administration include one or more intraosseous formulations and or one or more intraosseous delivery devices, while other methods of administration include one or more intrathecal formulations and or one or more intrathecal delivery devices.

In still further embodiments, the formulation of an at least one nitrogen-binding agent according to the present disclosure, and optionally ornithine and or ornithine benzoate, and or optionally including phenylacetate or phenylbutyrate or a derivative thereof, is administered to blood, cells, and or blood cells taken outside the body, and then the treated blood, cells, and or blood cells are placed back inside the body. Such methods can ensure that blood cells have adequate amounts the prodrug and or active ingredient. This may be useful when utilizing blood cells as bioreactors to facilitate these enzymatic reactions and ammonia scavenging properties. When loading the blood, cells, and or blood cells with these molecules outside the body, changes in osmotic potential can be employed to help ensure uptake by the cells and or blood cells. This example is not meant to be limiting.

Other formats and or combinations of formulations, and or other formats of delivery devices and or other routes of delivery of an at least one nitrogen-binding agent according to the present disclosure, and optionally ornithine and or ornithine benzoate are possible. The present disclosure is not intended to be limited to the embodiments shown herein, but is to be accorded the widest scope possible consistent with the principles and novel features as previously described.

Embodiments include immediate release formulations, while other embodiments include delayed or sustained release formulations.

Other embodiments include an at least one biotechnology formulation that includes one or more recombinant enzymes for injection, and include at least one of glutamine synthetase for injection, glutamine phenylacetyltransferase for injection, glycine benzoyltransferase for injection, or a combination thereof. These enzymes are found inside of cells. Glutamine synthetase is found inside the cytosol, while glutamine phenylacetyltransferase and glycine benzoyltransferase are mitochondrial enzymes. By injecting one or more of these recombinant enzymes into the bloodstream, the patient can experience a great increase in activity of these enzymes to produce more glutamine from glutamate while scavenging blood ammonia, and or to conjugate phenylacetate with glutamine, and or to conjugate glycine with benzoate, for faster elimination and ammonia clearance. The biotechnology formulation can include at least one excipient ingredient and or at least one solvent ingredient. The biotechnology formulation may include pegylated enzymes in some embodiments and or liposomes in some embodiments.

Other embodiments include methods of administering an at least one biotechnology formulation that includes one or more recombinant enzymes for injection, and of an at least one nitrogen-binding agent according to the present disclosure, and optionally ornithine and or ornithine benzoate, and or optionally phenylacetate or phenylbutyrate or a derivative thereof, including to a hyperammonemic patient.

In still further embodiments, an at least one biotechnology formulation includes one or more recombinant enzymes for injection, and include at least one of glutamine synthetase for injection, glutamine phenylacetyltransferase for injection, glycine benzoyltransferase for injection, or a combination thereof. In some embodiments, at least one of the recombinant enzymes for injection in the at least one biotechnology formulation are pegylated with polyethylene glycol. In some embodiments, at least one of the recombinant enzymes for injection in the at least one biotechnology formulation are provided in liposomes and or other vesicles for delivery. In some embodiments, this at least one biotechnology formulation is administered to blood, cells, and or blood cells taken outside the body, and then the treated blood, cells, and or blood cells are placed back inside the body. Such methods can ensure that blood cells have adequate amounts of the enzyme(s). This may be useful when utilizing blood cells as bioreactors to facilitate these enzymatic reactions and ammonia scavenging properties. When loading the blood, cells, and or blood cells with these molecules outside the body, changes in osmotic potential can be employed to help ensure uptake by the cells and or blood cells. This example is not meant to be limiting.

In still further embodiments, an at least one biotechnology formulation that includes one or more recombinant enzymes for injection, and include at least one of glutamine synthetase for injection, glutamine phenylacetyltransferase for injection, glycine benzoyltransferase for injection, or a combination thereof, also includes glutamate for injection, which may be in the form of monosodium glutamate, monopotassium glutamate, or even glutamyl-phosphate, or a salt, prodrug, derivative, or combination thereof. Other embodiments optionally also include at least one of phenylacetate, phenylbutyrate, phenylbutyrate ester, and or benzoate.

In still further embodiments, an at least one biotechnology formulation includes mRNA for injection to upregulate expression of one or more enzymes, including at least one of glutamine synthetase, glutamine phenylacetyltransferase, glycine benzoyltransferase, or a combination thereof. In other embodiments, interfering mRNA may be used to downregulate other enzymes and proteins.

The above embodiments are not meant to be limiting.

Other formats, preparations, formulations, and or combinations of formulations, and or other formats of delivery devices and or other routes of delivery and methods of administration of an at least one nitrogen-binding agent according to the present disclosure, and optionally ornithine and or ornithine benzoate, and optionally phenylacetate; and or further optionally, an at least one biotechnology formulation for injection or infusion that includes one or more recombinant enzymes for injection, including at least one of glutamine synthetase for injection, glutamine phenylacetyltransferase for injection, glycine benzoyltransferase for injection, or a combination thereof; are possible and the present disclosure is not intended to be limited to the embodiments shown herein, but is to be accorded the widest scope possible consistent with the principles and novel features as previously described.

### EXAMPLES

Example 1 is a pharmaceutical preparation comprising an at least one ester or amide of phenylbutyrate (i.e., an at least one phenylbutyrate ester and or phenylbutyrate amide) that does not hydrolyze into glycerol when it hydrolyzes to release phenylbutyrate molecules as a prodrug nitrogen-binding agent (i.e., a prodrug of the nitrogen-binding agent phenylacetate) for managing nitrogen levels, treating hyperammonemia, preventing hyperammonemia, treating hepatic encephalopathy, preventing hepatic encephalopathy, or a combination thereof. This generally also includes reducing hyperammonemia; i.e., reducing blood or plasma ammonia or ammonium levels. This can also include preventing the reoccurrence of hyperammonemia. In many examples, this pharmaceutical preparation is in liquid form. A liquid form may be desirable for administering large doses, such as for maintaining dosing compliance, and or for administration via feeding tubes. A liquid form can in some examples be administered via injection or infusion. In some examples, the liquid pharmaceutical preparation requires reconstitution or dilution with a solvent prior to use. In other examples, the phenylbutyrate ester(s) and or and or phenylbutyrate amide(s) are prepared as oral pellets, powders, capsules, and or tablets. In other examples, the pharmaceutical preparation is prepared as suppositories. Therefore, the pharmaceutical preparation may be a viscous liquid, a non-viscous liquid, a solid for reconstitution into liquid form, a solid for suspension into a liquid, or just a solid form. In many examples, hydrolysis generally takes place in the gastrointestinal tract of the patient, although hydrolysis may also take place in the bloodstream or organ(s) as well. In another example, the pharmaceutical preparation comprises diphenylbutyrate ester, i.e., two molecules of phenylbutyrate sharing the same ester bond and being covalently bound together. These are non-limiting examples.

Example 2, the subject matter of Example 1 additionally or alternatively includes, wherein said pharmaceutical preparation has no significant increase, or a much lower increase than glycerol, in at least one of triglyceride levels, lipoprotein particle levels, total cholesterol levels, body fat levels, or a combination thereof after hydrolysis and or absorption.

Example 3, the subject matter of any of Examples 1-2 additionally or alternatively includes, wherein said pharmaceutical preparation further includes at least one of ornithine, glutamate, glutamyl-phosphate, benzoate, ornithine benzoate ester, glutamate benzoate ester, phenylbutyrate benzoate ester, ornithine phenylbutyrate ester, glutamate phenylbutyrate ester, ornithine benzoate amide, glutamate benzoate amide, ornithine phenylbutyrate amide, glutamate phenylbutyrate amide, diphenylbutyrate ester, dibenzoate ester, or a combination thereof.

Example 4, the subject matter of any of Examples 1-3 additionally or alternatively includes, wherein said at least one phenylbutyrate ester and or phenylbutyrate amide is a molecule covalently bonded to at least one of benzoate, ornithine, glutamate, or a derivative, or combination thereof.

Example 5, the subject matter of any of Examples 1-4 additionally or alternatively includes, a pharmaceutical preparation comprising an at least one molecule that when hydrolyzed releases both an at least one phenylbutyrate molecule and an at least one molecule of at least one of benzoate, ornithine, glutamate, or a derivative, or combination thereof; the at least one phenylbutyrate molecule is released as a prodrug nitrogen-binding agent for managing nitrogen levels, treating hyperammonemia, preventing hyperammonemia, treating hepatic encephalopathy, preventing hepatic encephalopathy, or a combination thereof. In many examples, this pharmaceutical preparation is in liquid form. In some examples, the liquid pharmaceutical preparation requires reconstitution or dilution with a solvent prior to use. In other examples, the molecules are prepared as oral pellets, powders, capsules, and or tablets. In other examples, the pharmaceutical preparation is prepared as suppositories. Therefore, the pharmaceutical preparation may be a viscous liquid, a non-viscous liquid, a solid for reconstitution into liquid form, a solid for suspension into a liquid, or just a solid form. The at least one phenylbutyrate molecule can in some examples be covalently bonded to benzoate, ornithine, glutamate, or a derivative, or combination thereof directly, or indirectly via a linker (a linker of one or more atoms, e.g., atoms of carbon and or oxygen) that it is covalently bonded to. For example, the phenylbutyrate molecule can share directly an ester bond with benzoate, glutamate, ornithine, or a derivative thereof. In other examples, the phenylbutyrate is attached via an ester bond to an alcohol backbone, such as a glycol or even a triol (e.g., glycerol), while the benzoate, glutamate, ornithine, or a derivative thereof is also attached to the alcohol backbone; the alcohol backbone serving as a covalent linker. The benzoate, glutamate, ornithine, or a derivative thereof may be attached to the alcohol backbone via an ester bond, or indirectly via a linker of one or more atoms. These are non-limiting examples. In many examples, hydrolysis generally takes place in the gastrointestinal tract of the patient, but may in some instances occur in systemic circulation.

Example 6, the subject matter of any of Examples 1-5 additionally or alternatively includes, a pharmaceutical preparation comprising an at least one amide of phenylbutyrate (i.e., an at least one phenylbutyrate amide) that hydrolyzes to release phenylbutyrate molecules as a prodrug nitrogen-binding agent for managing nitrogen levels, treating hyperammonemia, preventing hyperammonemia, treating hepatic encephalopathy, preventing hepatic encephalopathy, or a combination thereof. This generally also includes reducing hyperammonemia; i.e., reducing blood or plasma ammonia or ammonium levels. This can also include preventing the reoccurrence of hyperammonemia. In many examples, this pharmaceutical preparation is in liquid form. In some examples, the liquid pharmaceutical preparation requires reconstitution or dilution with a solvent prior to use. In other examples, the phenylbutyrate amide(s) are prepared as oral pellets, powders, capsules, and or tablets. In other examples, the pharmaceutical preparation is prepared as suppositories. Therefore, the pharmaceutical preparation may be a viscous liquid, a non-viscous liquid, a solid for reconstitution into liquid form, a solid for suspension into a liquid, or just a solid form. The at least one amide of phenylbutyrate molecule can in some examples be covalently bonded to ornithine, glutamate, or a derivative, or combination thereof directly, or indirectly via a linker (e.g., a linker of one or more carbon atoms and or a nitrogen atom) that it is covalently bonded to. For example, the phenylbutyrate molecule can share directly an amide bond with glutamate, ornithine, or a derivative thereof. In other examples, the phenylbutyrate is attached to a nitrogen containing linker. These are non-limiting examples. In many examples, hydrolysis generally takes place in the gastrointestinal tract of the patient, but may in some instances occur in systemic circulation.

Example 7, the subject matter of any of Examples 1-6 additionally or alternatively includes, further comprising an amount of at least one milligram, a volume of at least one milliliter, a concentration of at least one milligram per milliliter, or a combination thereof, of said at least one molecule (e.g., of said at least one phenylbutyrate ester and or phenylbutyrate amide) that releases an at least one phenylbutyrate molecule when hydrolyzed.

Example 8, the subject matter of any of Examples 1-7 additionally or alternatively includes, further comprising an amount of at least one milligram, a volume of at least one milliliter, a concentration of at least one milligram per milliliter, or a combination thereof, of ornithine and or glutamate.

Example 9, the subject matter of any of Examples 1-8 additionally or alternatively includes, further comprising an amount of at least one milligram, a volume of at least one milliliter, a concentration of at least one milligram per milliliter, or a combination thereof, of benzoate, sodium benzoate, glutamate benzoate, and or ornithine benzoate.

Example 10, the subject matter of any of Examples 1-9 additionally or alternatively includes, ornithine and or ornithine benzoate in an equimolar ratio to said at least one ester of phenylbutyrate that does not hydrolyze into glycerol when it hydrolyzes to release phenylbutyrate molecules as a prodrug nitrogen-binding agent.

Example 11, the subject matter of any of Examples 1-10 additionally or alternatively includes, wherein said at least one ester of phenylbutyrate (i.e., said at least one phenylbutyrate ester) comprises a glycol (or diol) esterified to two 4-phenylbutanoate groups.

Example 12, the subject matter of any of Examples 1-11 additionally or alternatively includes, wherein said at least one ester of phenylbutyrate (i.e., said at least one phenylbutyrate ester) comprises a glycol (or diol) esterified to two 4-phenylbutanoate groups and wherein the glycol (or diol) is propylene glycol; 1,3-propanediol; 1,3-butanediol; or a combination thereof.

Example 13, the subject matter of any of Examples 1-12 additionally or alternatively includes, wherein said at least one ester of phenylbutyrate (i.e., said at least one phenylbutyrate ester) comprises a glycol (or diol) esterified to two 4-phenylbutanoate groups; said pharmaceutical preparation further including ornithine and or ornithine benzoate in a one-to-one (1:1) ratio with said at least one ester of phenylbutyrate.

Example 14, the subject matter of any of Examples 1-13 additionally or alternatively includes, wherein said at least one ester of phenylbutyrate (i.e., said at least one phenylbutyrate ester) comprises propane-1,2-diyl bis(4-phenylbutanoate); propane-1,3-diyl bis(4-phenylbutanoate); butane-1,3-diyl bis(4-phenylbutanoate); or a combination thereof.

Example 15, the subject matter of any of Examples 1-14 additionally or alternatively includes, wherein said at least one ester of phenylbutyrate (i.e., said at least one phenylbutyrate ester) comprises PEGylated phenylbutyrate and or PEGylated phenylbutyrate esters.

Example 16, the subject matter of any of Examples 1-15 additionally or alternatively includes, the pharmaceutical preparation further provided for oral administration.

Example 17, the subject matter of any of Examples 1-16 additionally or alternatively includes, the pharmaceutical preparation further provided for administration via a feeding tubing, gastrostomy tube or nasogastric tube.

Example 18, the subject matter of any of Examples 1-17 additionally or alternatively includes, the pharmaceutical preparation further provided in a multi-dose container.

Example 19, the subject matter of any of Examples 1-18 additionally or alternatively includes, further comprising an at least one excipient ingredient selected from at least one of flavoring agents, flavorants, sweeteners, taste-masking agents, solvents, tonicity agents, chelators, buffers, pH adjusting agents, solubility enhancing agents, absorption enhancing agents, cell membrane permeability enhancing agents, excipients for delaying release or delaying absorption, preservatives, or a combination thereof.

Example 20, the subject matter of any of Examples 1-19 additionally or alternatively includes, further comprising a nonaqueous solution.

Example 21, the subject matter of any of Examples 1-20 additionally or alternatively includes, further comprising an aqueous or semi-aqueous solution.

Example 22, the subject matter of any of Examples 1-21 additionally or alternatively includes, further including and or administered with pancreatic lipases.

Example 23, the subject matter of any of Examples 1-22 additionally or alternatively includes, further comprising and or administered with an at least one recombinant enzyme or enzyme expression system thereof, including at least one of glutamine synthetase, glutamine phenylacetyltransferase, glycine benzoyltransferase, or a combination thereof to increase an at least one enzymatic activity in the body. Enzyme expression systems are DNA- and or RNA-based expression systems coding for one or more of these enzymes. An example may include a DNA plasmid coding for one or more of these enzymes. Another example may include a viral vector (e.g., an adenovirus vector) containing the coding for one or more of these enzymes. Another example may include something similar to an RNA vaccine coding for one or more of these enzymes. These examples and ways of expressing these enzymes in the body are not meant to be limiting. The technology behind gene therapies may also be included to help increase expression of one or more of these enzymes. Expression of promoters of the genes coding for one or more of these enzymes may also be encompassed by this disclosure.

Example 24, the subject matter of any of Examples 1-23 additionally or alternatively includes, the pharmaceutical preparation further provided for injection.

Example 25, the subject matter of any of Examples 1-24 additionally or alternatively includes, the pharmaceutical preparation further provided for injection and comprising at least one sterile container selected from prefilled syringes, cartridges, autoinjectors, pen injectors, wearable injectors, portable infusion pump devices, vials, ampoules, intravenous fluid bags, and bottles.

Example 26, the subject matter of any of Examples 1-25 additionally or alternatively includes, further comprising no more than 10% impurities at release and or over its shelf life.

Example 27, the subject matter of any of Examples 1-26 additionally or alternatively includes, further having a shelf life of at least 12 months.

Example 28, the subject matter of any of Examples 1-27 additionally or alternatively includes, further comprising an at least one additional active ingredient used to treat a urea cycle disorder, liver disease, hyperammonemia, hepatic encephalopathy, or a combination thereof.

Example 29, the subject matter of any of Examples 1-28 additionally or alternatively includes, further comprising an immediate release format or formulation.

Example 30, the subject matter of any of Examples 1-29 additionally or alternatively includes, further comprising a delayed release and or extended release format or formulation. This may additionally or alternatively include a delayed absorption and or extended absorption formulation.

Further embodiments and examples are disclosed herein.

Example 31 is a pharmaceutical preparation comprising at least one of ornithine benzoate ester, glutamate benzoate ester, phenylbutyrate benzoate ester, ornithine phenylbutyrate ester, glutamate phenylbutyrate ester, ornithine benzoate amide, glutamate benzoate amide, ornithine phenylbutyrate amide, glutamate phenylbutyrate amide, or a combination thereof that further hydrolyzes to release phenylbutyrate molecules and or benzoate molecules as a nitrogen-binding agent for managing nitrogen levels, treating hyperammonemia, preventing hyperammonemia, treating hepatic encephalopathy, preventing hepatic encephalopathy, or a combination thereof. The pharmaceutical preparation can be made for oral administration or injection or infusion. In many examples, this pharmaceutical preparation is in liquid form. A liquid form may be desirable for administering large doses and or for administration via feeding tubes. A liquid form can in some examples be administered via injection or infusion. In some examples, a liquid pharmaceutical preparation requires reconstitution or dilution with a solvent prior to use. In other examples, the pharmaceutical preparation is prepared as oral pellets, powders, capsules, and or tablets. In other examples, the pharmaceutical preparation is prepared as suppositories. Therefore, the pharmaceutical preparation may be a viscous liquid, a non-viscous liquid, a solid for reconstitution into liquid form, a solid for suspension into a liquid, or just a solid form. In other embodiments, phenylacetate (instead of phenylbutyrate) is covalently bound directly, or indirectly through one or more linker atoms, to at least one of ornithine, benzoate, glutamate, or a derivative or combination thereof, and these embodiments include an at least one pharmaceutical preparation of such for injection or oral administration or as a suppository.

Example 32 is an injectable liquid pharmaceutical preparation comprising ornithine benzoate and optionally sodium phenylacetate and an at least one excipient ingredient.

Still further embodiments and examples are disclosed herein.

Example 33 is a method comprising administering an at least one dosage of an at least one pharmaceutical preparation comprising an at least one amide and or ester of phenylbutyrate (i.e., an at least one phenylbutyrate amide and or an at least one phenylbutyrate ester), other than the molecule 2,3-bis(4-phenylbutanoyloxy)propyl 4-phenylbutanoate [the molecule known as glycerol phenylbutyrate], that hydrolyzes to release phenylbutyrate molecules as a prodrug nitrogen-binding agent for managing nitrogen levels, treating hyperammonemia, preventing hyperammonemia, treating hepatic encephalopathy, preventing hepatic encephalopathy, or a combination thereof. In some embodiments, this example includes other glycerol backbone-containing molecules, just not 2,3-bis(4-phenylbutanoyloxy)propyl 4-phenylbutanoate. In many embodiment examples, the least one pharmaceutical preparation comprising an at least one amide and or ester of phenylbutyrate, other than the molecule 2,3-bis(4-phenylbutanoyloxy)propyl 4-phenylbutanoate, does not hydrolyze into glycerol when it hydrolyzes to release phenylbutyrate molecules as a prodrug nitrogen-binding agent; while in other embodiments, it can hydrolyze into glycerol. In many embodiment examples, the at least one pharmaceutical preparation is in liquid form, although other examples may include solid oral forms and suppositories. Therefore, the pharmaceutical preparation may be a viscous liquid, a non-viscous liquid, a solid for reconstitution into liquid form, a solid for suspension into a liquid, or just a solid form.

Example 34, the subject matter of Example 33 additionally or alternatively includes, wherein said an at least one pharmaceutical preparation is further provided for oral administration as an oral dosage form.

Example 35, the subject matter of any of Examples 33-34 additionally or alternatively includes, wherein said an at least one pharmaceutical preparation is further provided for administration via a feeding tubing, gastrostomy tube or nasogastric tube.

Example 36, the subject matter of any of Examples 33-35 additionally or alternatively includes, wherein said an at least one pharmaceutical preparation is further provided for injection.

Example 37, the subject matter of any of Examples 33-36 additionally or alternatively includes, wherein said an at least one pharmaceutical preparation is further provided for injection, and wherein said administering is selected from intravenous bolus injection, intravenous bolus infusion, continuous intravenous infusion, subcutaneous injection, intramuscular injection, intrathecal injection, intraosseous injection, autoinjection, implantation, or a combination thereof.

Example 38, the subject matter of any of Examples 33-37 additionally or alternatively includes, wherein said method further includes administering at least one of ornithine, benzoate, ornithine benzoate, sodium benzoate, glutamate, glutamate benzoate, glutamyl-phosphate, or a salt, prodrug, derivative, or combination thereof with the same formulation or separately; and or alternatively, said at least one pharmaceutical preparation further comprises at least one of ornithine, benzoate, ornithine benzoate, sodium benzoate, glutamate, glutamate benzoate, glutamyl-phosphate, or a salt, prodrug, derivative, or combination thereof.

Example 39, the subject matter of any of Examples 33-38 additionally or alternatively includes, wherein levels of circulating phenylacetate and or phenylacetylglutamine increase by more than 1 percent in a body of a patient following administration; and optionally includes measuring said level.

Example 40, the subject matter of any of Examples 33-39 additionally or alternatively includes, wherein levels of phenylacetylglutamine excreted by the kidneys increase by more than 1 percent in a body of a patient following administration; and optionally includes measuring said level.

Example 41, the subject matter of any of Examples 33-40 additionally or alternatively includes, wherein levels of circulating ammonia and or ammonium ion decrease by more than 1 percent in a body of a patient following administration; and optionally includes measuring said level.

Example 42, the subject matter of any of Examples 33-41 additionally or alternatively includes, further comprising administering at least two or more dosages of said an at least one pharmaceutical preparation to a patient or subject.

Example 43, the subject matter of any of Examples 33-42 additionally or alternatively includes, further comprising reconstituting or diluting said an at least one pharmaceutical preparation with a solvent prior to use.

Example 44, the subject matter of any of Examples 33-43 additionally or alternatively includes, further comprising administering said an at least one pharmaceutical preparation to a patient with a urea cycle disorder.

Example 45, the subject matter of any of Examples 33-44 additionally or alternatively includes, further comprising administering said an at least one pharmaceutical preparation to a patient with an inborn error of metabolism, enzyme deficiency, and or enzyme inefficiency.

Example 46, the subject matter of any of Examples 33-45 additionally or alternatively includes, further comprising administering said an at least one pharmaceutical preparation to a patient with an acute liver disease or an acute liver failure.

Example 47, the subject matter of any of Examples 33-46 additionally or alternatively includes, further comprising administering said an at least one pharmaceutical preparation to a patient with a chronic liver disease or a chronic liver failure or liver cirrhosis or liver transplant.

Example 48, the subject matter of any of Examples 33-47 additionally or alternatively includes, further comprising administering said an at least one pharmaceutical preparation to a patient having high levels of at least one of triglycerides, lipoprotein particles, cholesterol, body fat, or a combination thereof, and or the patient has or is close to having at least one of heart disease, cardiovascular disease, atherosclerosis, lipid storage disease, or a combination thereof; for which glycerol intake should be reduced, restricted, or contraindicated. It was found that glycerol should not be administered to such patients, or that glycerol should be contraindicated for such patients, as glycerol (e.g., such as the glycerol released in the medication Ravicti) can exacerbate these levels and or conditions. For example, patients with liver problems or liver dysfunction often have irregular lipid, cholesterol, and or lipoprotein levels; and liver dysfunction can often lead to elevated blood ammonia levels and or hyperammonemia. For such patients, the many phenylbutyrate esters disclosed in the above embodiments would be ideal, as these phenylbutyrate esters do not supply glycerol to the body.

Example 49, the subject matter of any of Examples 33-48 additionally or alternatively includes, further comprising administering said an at least one pharmaceutical preparation to a patient taking a drug, or exposed to a toxin, that causes hyperammonemia.

Example 50, the subject matter of any of Examples 33-49 additionally or alternatively includes, further comprising administering said an at least one pharmaceutical preparation to a patient having acidosis or alkalosis. Alternatively or additionally, the patient may have an infection or sepsis or septic shock.

Example 51, the subject matter of any of Examples 33-50 additionally or alternatively includes, further comprising administering said an at least one pharmaceutical preparation to a patient taking oral ammonia scavenging medication that failed to manage ammonia levels.

Example 52, the subject matter of any of Examples 33-51 additionally or alternatively includes, further comprising administering said an at least one pharmaceutical preparation to a patient to prevent hemodialysis, reduce hemodialysis time, reduce hemodialysis frequency, or a combination thereof in a patient with hyperammonemia and or hepatic encephalopathy.

Example 53, the subject matter of any of Examples 33-52 additionally or alternatively includes, further comprising administering said an at least one pharmaceutical preparation to a patient to prevent or reduce symptoms of hyperammonemia and or hepatic encephalopathy.

Example 54, the subject matter of any of Examples 33-53 additionally or alternatively includes, further comprising administering said an at least one pharmaceutical preparation to a patient to prevent or reduce neurological damage or cognitive damage associated with hyperammonemia and or hepatic encephalopathy.

Example 55, the subject matter of any of Examples 33-54 additionally or alternatively includes, further comprising administering said an at least one pharmaceutical preparation to a newborn patient having or suspected of having hyperammonemia and or hepatic encephalopathy.

Example 56, the subject matter of any of Examples 33-55 additionally or alternatively includes, further comprising administering said an at least one pharmaceutical preparation to a child patient having or suspected of having hyperammonemia and or hepatic encephalopathy.

Example 57, the subject matter of any of Examples 33-56 additionally or alternatively includes, further comprising administering said an at least one pharmaceutical preparation to an adult patient having or suspected of having hyperammonemia and or hepatic encephalopathy.

Example 58, the subject matter of any of Examples 33-57 additionally or alternatively includes, further comprising administering said an at least one pharmaceutical preparation to a patient having or suspected of having deficiency in glutamate, glutamine, ornithine, or a combination thereof.

Example 59, the subject matter of any of Examples 33-58 additionally or alternatively includes, further comprising the steps of measuring blood or plasma ammonia or ammonium levels at least once before and or at least once after administering said an at least one dosage of said pharmaceutical preparation to said patient at least once; said method optionally modifying the administration and or an amount of said an at least one dosage accordingly.

Example 60, the subject matter of any of Examples 33-59 additionally or alternatively includes, further comprising the steps of measuring blood or plasma ammonia or ammonium levels before and after administering said an at least one pharmaceutical preparation, along with the step of measuring blood or plasma ammonia or ammonium levels after administering said an at least one pharmaceutical preparation a second time; said method optionally modifying the administration and or an amount of said an at least one dosage accordingly.

Example 61, the subject matter of any of Examples 33-60 additionally or alternatively includes, further comprising the steps of measuring blood or plasma ammonia or ammonium levels before each step of administering said an at least one pharmaceutical preparation.

Example 62, the subject matter of any of Examples 33-61 additionally or alternatively includes, further comprising the steps of measuring blood or plasma ammonia or ammonium levels after each step of administering said an at least one pharmaceutical preparation.

Example 63, the subject matter of any of Examples 33-62 additionally or alternatively includes, further comprising the step of hemodialysis before, during, and or after the administering said an at least one pharmaceutical preparation.

Example 64, the subject matter of any of Examples 33-63 additionally or alternatively includes, further comprising gene or genetic sequencing or detecting genetic mutations in, and or measuring levels of, and or measuring levels of activity of, one or more enzymes involved in and or related to the metabolism of glutamate, glutamine, ornithine, alpha-ketoglutarate, ammonia, urea, benzoate, phenylacetate, or a combination thereof; said method optionally modifying the administration and or an amount of said an at least one dosage accordingly.

Example 65, the subject matter of any of Examples 33-64 additionally or alternatively includes, further comprising gene or genetic sequencing or detecting genetic mutations in, and or measuring levels of, and or measuring levels of activity of, one or more enzymes involved in and or related to the metabolism of glutamate, glutamine, ornithine, alpha-ketoglutarate, ammonia, urea, benzoate, phenylacetate, or a combination thereof, and further comprising modifying the administration and or an amount of said an at least one pharmaceutical preparation accordingly.

Example 66, the subject matter of any of Examples 33-65 additionally or alternatively includes, further comprising administering an at least one biotechnology formulation that includes one or more recombinant enzymes, including at least one of glutamine synthetase, glutamine phenylacetyltransferase, glycine benzoyltransferase, or a combination or expression system (DNA- and or RNA-based expression systems coding for one or more of these enzymes) thereof.

Example 67, the subject matter of any of Examples 33-66 additionally or alternatively includes, wherein said an at least one pharmaceutical preparation further comprises an at least one biotechnology formulation that includes one or more recombinant enzymes, including at least one of glutamine synthetase, glutamine phenylacetyltransferase, glycine benzoyltransferase, or a combination or expression system (DNA- and or RNA-based expression systems coding for one or more of these enzymes) thereof.

Still further embodiments and examples are disclosed herein.

Example 68 is an at least one biotechnology formulation for injection or infusion that comprises one or more recombinant enzymes for injection, including at least one of glutamine synthetase for injection, glutamine phenylacetyltransferase for injection, glycine benzoyltransferase for injection, or a combination thereof, and at least one excipient ingredient.

Example 69, the subject matter of Example 68 additionally or alternatively includes, further comprising at least one substrate or active ingredient including at least one of glutamate, glutamyl-phosphate, phenylacetate, phenylbutyrate, benzoate, ornithine, ornithine benzoate ester or amide, glutamate benzoate ester or amide, phenylbutyrate ester, phenylbutyrate amide, or a salt, prodrug, derivative, or combination thereof.

Example 70, the subject matter of any of Examples 68-69 additionally or alternatively includes, further comprising a liposomal and or pegylated form of the one or more recombinant enzymes for injection.

Example 71 is a method comprising administering an at least one biotechnology formulation for injection or infusion that comprises an at least one recombinant enzyme for injection, including at least one of glutamine synthetase for injection, glutamine phenylacetyltransferase for injection, glycine benzoyltransferase for injection, or a combination thereof, for treating hyperammonemia, preventing hyperammonemia, treating hepatic encephalopathy, preventing hepatic encephalopathy, or a combination thereof; said method optionally including a step of extracorporeal administration of said at least one biotechnology formulation to blood cells and or therapeutic apheresis.

Example 72, the subject matter of Example 71 additionally or alternatively includes, further including the step of administering at least one substrate or active ingredient including at least one of glutamate, glutamyl-phosphate, phenylacetate, phenylbutyrate, benzoate, ornithine, ornithine benzoate ester or amide, glutamate benzoate ester or amide, phenylbutyrate ester, phenylbutyrate amide, or a salt, prodrug, derivative, or combination thereof.

Example 73, the subject matter of any of Examples 71-72 additionally or alternatively includes, further including the step of removing some blood or blood cells, then administering the at least one biotechnology formulation for injection or infusion to the blood or blood cells removed from a body of a patient, and further including the step of injecting or infusing the treated blood or blood cells back into a body of a patient. This may also include administering any one of the pharmaceutical preparations described in previous examples above to the blood cells removed from the body during treatment with the biotechnology treatment and placed back in the body; and or administering any one of the pharmaceutical preparations described in previous examples above after performing said biotechnology treatment above.

The above examples are not meant to be limiting.

Glutamic acid is sometimes included in intravenous parenteral nutrition amino acid solutions containing over a dozen other amino acids for injection, including essential amino acids. These amino acid solutions are generally indicated to provide a source of nitrogen in the nutritional support of patients, including for newborns. The glutamic acid contained in these parenteral nutrition products are generally supplied in a concentration of about 5 mg per milliliter. Important distinctions exist to avoid confusion between these parenteral nutrition amino acid solutions that contain glutamate and other amino acids as a source of nitrogen, which can increase ammonia or ammonium levels, and the glutamate for injection in some examples of the present disclosure (e.g., generally supplied at a concentration of about 100 mg per milliliter or so) for treating hyperammonemia for use with or containing phenylacetate or phenylbutyrate.

In the embodiments and examples described above, preparations and or formulations, including injectable preparations and formulations and oral preparations and formulations, are generally meant to be pharmaceutically acceptable, and as such, are generally safe, non-toxic, and therapeutically desirable for use in human and or veterinary patients.

Various illustrative components, blocks, configurations, modules, and steps have been described above generally in terms of their functionality. Persons having ordinary skill in the art may implement the described functionality in varying ways for each particular application, but such implementation decisions should not be interpreted as causing a departure from the scope of the present disclosure.

The previous description of the disclosed embodiments is provided to enable a person skilled in the art to make or use the disclosed embodiments. Various modifications to these embodiments will be readily apparent to those skilled in the art, and the principles defined herein may be applied to other embodiments without departing from the scope of the disclosure. Thus, the present disclosure is not intended to be limited to the embodiments shown herein but is to be accorded the widest scope possible consistent with the principles and novel features as previously described.

1. A pharmaceutical preparation comprising at least one of: a phenylbutyrate ester or a phenylbutyrate amide, wherein the phenylbutyrate ester or the phenylbutyrate amide does not hydrolyze into glycerol during hydrolyzation to release phenylbutyrate molecules as a prodrug nitrogen-binding agent for managing nitrogen levels, treating hyperammonemia, preventing hyperammonemia, treating hepatic encephalopathy, preventing hepatic encephalopathy, or a combination thereof.

2. The pharmaceutical preparation of claim 1, wherein said pharmaceutical preparation increases a receiving body's levels of at least one of triglyceride, lipoprotein particle, total cholesterol, body fat, or a combination thereof after hydrolysis by an amount less than an increase caused by an application of glycerol to the body.

3. The pharmaceutical preparation of claim 1, further comprising at least one of ornithine, glutamate, glutamyl-phosphate, benzoate, ornithine benzoate ester, glutamate benzoate ester, phenylbutyrate benzoate ester, ornithine phenylbutyrate ester, glutamate phenylbutyrate ester, ornithine benzoate amide, glutamate benzoate amide, ornithine phenylbutyrate amide, glutamate phenylbutyrate amide, diphenylbutyrate ester, or dibenzoate ester.

4. The pharmaceutical preparation of claim 1, wherein the phenylbutyrate ester and or phenylbutyrate amide is a molecule covalently bonded to at least one of benzoate, ornithine, glutamate, or a derivative, or combination thereof.

5. A pharmaceutical preparation comprising:
at least one molecule that when hydrolyzed releases both:
at least one phenylbutyrate molecule and
at least one molecule, wherein the at least one molecule is at least one of benzoate, ornithine, glutamate, or a derivative, or combination thereof;
wherein the at least one phenylbutyrate molecule is released as a prodrug nitrogen-binding agent for managing nitrogen levels, treating hyperammonemia, preventing hyperammonemia, treating hepatic encephalopathy, preventing hepatic encephalopathy, or a combination thereof.

6. A pharmaceutical preparation comprising at least one phenylbutyrate amide that hydrolyzes to release phenylbutyrate molecules as a prodrug nitrogen-binding agent for managing nitrogen levels, treating hyperammonemia, preventing hyperammonemia, treating hepatic encephalopathy, preventing hepatic encephalopathy, or a combination thereof.

7. The pharmaceutical preparation of claim 1, wherein the at least one of: a phenylbutyrate ester or a phenylbutyrate amide is provided in an amount of at least one milligram, a volume of at least one milliliter, a concentration of at least one milligram per milliliter, or a combination thereof, of said at least one phenylbutyrate ester and or phenylbutyrate amide.

8. The pharmaceutical preparation of claim 1 comprising the at least one phenylbutyrate ester, wherein said at least one phenylbutyrate ester comprises a glycol (or diol) esterified to two 4-phenylbutanoate groups.

9. The pharmaceutical preparation of claim 1, comprising the at least one phenylbutyrate ester, wherein said at least one phenylbutyrate ester comprises propane-1,2-diyl bis(4-phenylbutanoate); propane-1,3-diyl bis(4-phenylbutanoate); butane-1,3-diyl bis(4-phenylbutanoate); or a combination thereof.

10. The pharmaceutical preparation of claim 1, comprising the at least one phenylbutyrate ester, wherein said at least one phenylbutyrate ester comprises PEGylated phenylbutyrate and or PEGylated phenylbutyrate esters.

11. The pharmaceutical preparation of claim 1 provided as a composition for one of: oral administration, administration via a feeding tubing, administration via a gastrostomy tube, administration via a nasogastric tube, or rectal administration.

12. The pharmaceutical preparation of claim 1 further comprising at least one excipient ingredient selected from: flavoring agents, flavorants, sweeteners, taste-masking agents, solvents, tonicity agents, chelators, buffers, pH adjusting agents, solubility enhancing agents, absorption enhancing agents, cell membrane permeability enhancing agents, excipients for delaying release or delaying absorption, preservatives, or a combination thereof.

13. The pharmaceutical preparation of claim 1 further comprising at least one recombinant enzyme or enzyme expression system thereof, including at least one of glutamine synthetase, glutamine phenylacetyltransferase, glycine benzoyltransferase, or a combination thereof, to increase an at least one enzymatic activity in a patient's body.

14. The pharmaceutical preparation of claim 1 provided as a composition for injection, and wherein the pharmaceutical preparation comprises at least one sterile container selected from prefilled syringes, cartridges, autoinjectors, pen injectors, wearable injectors, portable infusion pump devices, vials, ampoules, intravenous fluid bags, and bottles.

15. The pharmaceutical preparation of claim 1 further comprising at least one additional active ingredient used to treat a urea cycle disorder, liver disease, hyperammonemia, hepatic encephalopathy, or a combination thereof.

16. A pharmaceutical preparation comprising at least one of ornithine benzoate ester, glutamate benzoate ester, phenylbutyrate benzoate ester, ornithine phenylbutyrate ester, glutamate phenylbutyrate ester, ornithine benzoate amide, glutamate benzoate amide, ornithine phenylbutyrate amide, glutamate phenylbutyrate amide, or a combination thereof that hydrolyzes to release one or more of: (a) phenylbutyrate molecules or (b) benzoate molecules as a nitrogen-binding agent for managing nitrogen levels, treating hyperammonemia, preventing hyperammonemia, treating hepatic encephalopathy, preventing hepatic encephalopathy, or a combination thereof.

17. A method, comprising:
administering at least one dosage of at least one pharmaceutical preparation comprising at least one of: a phenylbutyrate amide or a phenylbutyrate ester other than 2,3-bis(4-phenylbutanoyloxy)propyl 4-phenylbutanoate that hydrolyzes to release phenylbutyrate molecules as a prodrug nitrogen-binding agent for managing nitrogen levels, treating hyperammonemia, preventing hyperammonemia, treating hepatic encephalopathy, preventing hepatic encephalopathy, or a combination thereof;
wherein said method causes at least one of:
levels of circulating phenylacetate or phenylacetylglutamine to increase by more than 1 percent in a body of a patient following administration,
levels of phenylacetylglutamine excreted by kidneys in the body of the patient to increase by more than 1 percent in the body of the patient following administration, or
levels of circulating ammonia or ammonium ion to decrease by more than 1 percent in the body of the patient following administration.

18. The method of claim 17, further comprising administering at least one of ornithine, benzoate, ornithine benzoate, sodium benzoate, glutamate, glutamate benzoate, glutamyl-phosphate, or a salt, prodrug, derivative, or combination thereof in said at least one pharmaceutical preparation or separately.

19. The method of claim 17, further comprising administering said at least one dosage to a patient with a urea cycle disorder; to a patient with an inborn error of metabolism, enzyme deficiency, and or enzyme inefficiency; to a patient with an acute liver disease or an acute liver failure; to a patient with a chronic liver disease or a chronic liver failure or liver cirrhosis or liver transplant; or a combination thereof.

20. The method of claim 17, further comprising administering said at least one dosage to a patient, wherein the patient satisfies one or more of:
(a) the patient has high levels of at least one of triglycerides, lipoprotein particles, cholesterol, body fat, or a combination thereof; or
(b) the patient has or is close to having at least one of heart disease, cardiovascular disease, atherosclerosis, lipid storage disease, or a combination thereof; for which glycerol intake should be reduced, restricted, or contraindicated.

21. The method of claim 17, further comprising steps of measuring blood or plasma ammonia or ammonium levels at least once before or at least once after administering said an at least one dosage to said patient at least once.

22. The method of claim 17, further comprising one or more of:
sequencing a patient's genes;
detecting genetic mutations in the patient; or
measuring levels of activity of one or more enzymes involved in and or related to metabolism of glutamate, glutamine, ornithine, alpha-ketoglutarate, ammonia, urea, benzoate, phenylacetate, or a combination thereof.

23. The method of claim 17, further comprising administering at least one biotechnology formulation that includes one or more recombinant enzymes, including at least one of glutamine synthetase, glutamine phenylacetyltransferase, glycine benzoyltransferase, or a combination or expression system thereof.

24. The method of claim 17, wherein said an at least one pharmaceutical preparation further comprises at least one biotechnology formulation that includes one or more recombinant enzymes, including at least one of glutamine synthetase, glutamine phenylacetyltransferase, glycine benzoyltransferase, or a combination or expression system thereof.

25. A biotechnology formulation for injection or infusion comprising one or more recombinant enzymes for injection, including at least one of glutamine synthetase for injection, glutamine phenylacetyltransferase for injection, glycine benzoyltransferase for injection, or a combination thereof, and at least one excipient ingredient.

26. A method, comprising:
administering at least one biotechnology formulation for injection or infusion that comprises at least one recombinant enzyme for injection, including at least one of glutamine synthetase for injection, glutamine phenylacetyltransferase for injection, glycine benzoyltransferase for injection, or a combination thereof, for treating hyperammonemia, preventing hyperammonemia, treating hepatic encephalopathy, preventing hepatic encephalopathy, or a combination thereof.

27. The method of claim 26, further comprising a step of administering at least one substrate or active ingredient including at least one of glutamate, glutamyl-phosphate, phenylacetate, phenylbutyrate, benzoate, ornithine, ornithine benzoate ester or amide, glutamate benzoate ester or amide, phenylbutyrate ester, phenylbutyrate amide, or a salt, prodrug, derivative, or combination thereof.
